(19) United States
(12) Patent Application Publication
(10) Pub. No.: US 2023/0282331 A1
(43) Pub. Date: Sep. 7, 2023

(54) Virtual Reality Eating Behavior Training Systems and Methods

(71) Applicants: Per SÖDERSTEN, Stockholm (SE); Billy Sundström LANGLET, Bromma (SE); Michael LEON, San Juan Capistrano, CA (US); Cecilia BERGH, Stockholm (SE); Mandometer AB, Huddinge (SE)

(72) Inventors: Cecilia Bergh, Stockholm (SE); Per Södersten, Stockholm (SE); Billy Sundström LANGLET, Bromma (SE); Michael LEON, San Juan Capistrano, CA (US)

(21) Appl. No.: 17/999,433

(22) PCT Filed: May 27, 2021
(86) PCT No.: PCT/US21/34418

§ 371 Date: Nov. 21, 2022

Related U.S. Application Data
(60) Provisional application No. 63/031,239, filed on May 28, 2020.

(51) Int. Cl. G16H 20/60; G16H 50/20; A61B 5/00

(52) U.S. Cl. CPC G16H 20/60; G16H 50/20; A61B 5/486

(57) ABSTRACT

Virtual reality (VR) systems and methods for treating eating disorders or training a patient to have beneficial eating habits. Systems includes a virtual reality interface displayed to a patient and a computing system coupled to the virtual reality interface. The computing system executes instructions to evaluate a patient eating disorder based upon patient input and to construct and display a therapeutic VR environment, where the patient can be treated for the eating disorder and trained to have beneficial eating habits.

### CROSS-REFERENCES TO RELATED APPLICATIONS

This application claims priority to U.S. Patent Application Ser. No. 63/031,239 (the “'239 Application”), filed Nov. 18, 2019, by Cecilia Bergh et al. (attorney docket no. 1115.06PR), entitled, “Virtual Reality Eating Behavior Training System” the disclosure of which is incorporated herein by reference in its entirety for all purposes.

This application may be related to U.S. patent application Ser. No. 12/412,434 (the “'434 Application”; U.S. Pat. No. 10,332,054), filed Mar. 27, 2009 by Cecilia Bergh et al. (attorney docket no. 1115.02), entitled, “Method, Generator Device, Computer Program Product and System for Generating Medical Advice,” which claims priority to SE Application No. 0900156-1, filed Feb. 9, 2009, by Cecilia Bergh, the disclosure of which is incorporated herein by reference in its entirety for all purposes. This application may also be related to International Application No. PCT/US20/58248 (the “'248 Application”), filed Oct. 2, 2020, by Cecilia Bergh et al. (attorney docket no. 1115.04PCT), entitled, “Eating Disorder Diagnosis,” the disclosure of which is incorporated herein by reference in its entirety for all purposes. This application may also be related to U.S. patent application Ser. No. 16/837,456 (the “'456 Application”; U.S. Publication No. US 2020/0222658), filed Apr. 1, 2020, by Michael Leon, entitled “Cognition and Memory Enhancement Via Multiple Odorant Stimulation,” the disclosure of which is incorporated herein by reference in its entirety for all purposes.

The respective disclosures of these applications/patents (which this document refers to collectively as the “Related Applications”) are incorporated herein by reference in their entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates to methods, systems, and apparatuses for eating behavior training, and, more particularly, to methods, systems, and apparatuses for implementing eating behavior training using a virtual reality (VR) interface and programming.

### BACKGROUND

Eating disorders include a range of psychological conditions that cause unhealthy, detrimental, or destructive eating habits to develop. An eating disorder might start with an obsession with food, body weight, or body shape. Eating disorders include, but are not limited to anorexia, bulimia, binge eating disorders, pica, and other disorders. Eating disorders can be associated with other habits or activities, including obsessive exercising or social avoidance. Eating disorders can be physically and emotionally damaging or fatal if left untreated.

Patients with eating disorders have traditionally been treated with psychological counseling, nutritional counseling, cognitive behavioral therapy, and monitoring of patient behavior. Conventional treatments have required patients to be evaluated, counseled, and monitored in a clinical setting, limiting the ways in which treatment is provided by a healthcare professional, and received by the patient.

Various studies have shown that a virtual reality (VR) environment can be used to treat conditions such as post-traumatic stress syndrome, anxiety syndrome, and nicotine addiction. With the use of a VR computer program and associated VR hardware, a patient can be immersed in a three-dimensional world customized according to their therapeutic needs and can be safely exposed to stressors or treatments. Hence, there is a need for robust and scalable VR solutions for treating eating disorders or training a patient to have beneficial eating habits.

### BRIEF DESCRIPTION OF THE DRAWINGS

A further understanding of the nature and advantages of particular embodiments may be realized by reference to the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic block diagram of a VR eating behavior training system, in accordance with various embodiments.

FIG. 2 is a flow diagram of a method for VR eating behavior training, in accordance with some embodiments.

FIG. 3 is a flow diagram of a method for VR eating behavior training, featuring artificial intelligence and/or machine learning analysis.

FIG. 4 is a flow diagram of a method for VR eating behavior training, featuring multiple outputs to create a virtual environment.

FIG. 5 is a flow diagram of a method for VR eating behavior training, featuring autonomous biofeedback input to the system.

FIG. 6 is a flow diagram of a method for VR eating behavior training, featuring real time progress feedback and remission assessment.

FIG. 7 is a flow diagram of a method for VR eating behavior training, featuring selected virtual elements.

FIG. 8 illustrates a VR interface presenting a table setting to initiate eating by successive approximations in the treatment of anorexic behavior, in accordance with various embodiments.

FIG. 9 illustrates a VR interface presenting a serving of water to initiate drinking and eating in the treatment of anorexic behavior, in accordance with various embodiments.

FIG. 10 illustrates a VR interface presenting a serving of food on a plate to initiate eating in the treatment of anorexic behavior, in accordance with various embodiments.

FIG. 11 illustrates a VR interface presenting a staircase as non-rewarding stimulus in the treatment of anorexic behavior, in accordance with various embodiments.

FIG. 12 illustrates a VR interface presenting a list of “forbidden foods” in the treatment of eating disorders, in accordance with various embodiments.

FIG. 13 illustrates a VR interface presenting an assisted “forbidden food” training scenario in the treatment of eating disorders, in accordance with various embodiments.

FIG. 14 illustrates a VR interface presenting an unassisted “forbidden food” training scenario in the treatment of eating disorders, in accordance with various embodiments.

FIG. 15 illustrates a VR interface presenting a food selection training scenario, in accordance with various embodiments.

FIG. 16 illustrates a VR interface presenting a dessert food selection training scenario, in accordance with various embodiments.

FIG. 17 illustrates a VR interface presenting a food selection scenario using two “forbidden foods” with a model guide, in accordance with various embodiments.

FIG. 18 illustrates a VR interface presenting a normal eating behavior scenario, in accordance with various embodiments.

FIG. 19 illustrates a VR interface presenting a risk-taking scenario, in accordance with various embodiments.

FIG. 20 illustrates a VR interface presenting a testing scenario for remission confirmation, in accordance with various embodiments.

FIG. 21 is a schematic block diagram of a computer system for VR eating behavior training, in accordance with various embodiments.

FIG. 22 is a schematic block diagram illustrating a system of networked computer devices, in accordance with various embodiments.

### DETAILED DESCRIPTION OF CERTAIN EMBODIMENTS

Overview

Various embodiments provide robust and scalable VR solutions for treating eating disorders or training a patient to have beneficial eating habits. The following detailed description illustrates a few exemplary embodiments in further detail to enable one of skill in the art to practice such embodiments. The described examples are provided for illustrative purposes and are not intended to limit the scope of the invention.

Embodiments disclosed herein include a system of one or more computers and associated hardware configured to perform or implement certain virtual reality eating behavior training methods. System or apparatus embodiments may further include one or more computer programs stored on a tangible medium that can be configured to perform operations or actions by virtue of instructions that, when executed by data processing apparatus, cause the apparatus to perform the methods.

One general aspect disclosed herein includes a virtual reality system configured to display a virtual reality interface to a patient and a computing system coupled to the virtual reality interface. The computing system may include a processor and a computer readable medium in communication with the processor. The computer readable medium will have encoded thereon a set of instructions executable by the processor to take one or more of the following actions:
1) accept an input of one or more patient eating behaviors;
2) evaluate the input of one or more patient eating behaviors to determine a patient eating disorder;
3) determine a target eating behavior based, at least in part, on the patient eating disorder;
4) generate a virtual scenario for training the patient to accomplish the target eating behavior;
5) present the virtual scenario to the patient through the virtual reality interface, where the virtual scenario allows the patient to virtually accomplish the target eating behavior;
6) monitor one or more patient actions within the virtual scenario through the virtual reality interface; and
7) provide real-time feedback to the patient, through the virtual reality interface, based on the one or more patient actions within the virtual scenario, said real-time feedback being configured to encourage the target eating behavior.

Specific system implementations may include optional features. for example, the system may be configured to utilize artificial intelligence functionality or machine learning functionality to perform an evaluation step including but not limited to a) evaluating the input of one or more patient eating behaviors; b) determining the patient eating disorder; and c) determining the target eating behavior.

In some embodiments, the virtual reality interface may include a visual output, an audible output and at least one of a tactile and an olfactory output. The virtual reality interface may include at least one biofeedback detector. The biofeedback detector(s) may include some or all of an eye motion detector, a pupil dilation detector, a blink rate detector, a skin temperature detector, a perspiration level detector, a breathing rate detector, a cardiac pulse rate detector, a blood oxygen level detector, a blood pressure detector with similar detectors or sensors.

The virtual scenario presented by the system may include one or more successive eating approximations configured to initiate the target eating behavior in the patient. The one or more successive eating approximations may include at least one of setting a table, pouring a drink, placing a food on a plate, picking up the food with a fork, and smelling the food.

The system may be configured generate, from the patient input a list of “fear” foods or “forbidden” foods. In such an implementation, the virtual scenario may be generated, at least in part, utilizing the list of fear or forbidden foods from the patient. The virtual scenario may include a food selection scenario configured to allow the patient to virtually select at least one item from the list of fear or forbidden foods for consumption. The virtual scenario may be configured to present one or more virtual human guides to the patient within the virtual scenario, where the one or more virtual human guides model the target eating behavior within the virtual scenario. The system may further be configured to determine, based on the one or more patient actions, whether the patient eating disorder is in remission. Any virtual scenario generated by the system may present a stimulus to the patient to elicit a reward response.

Other aspects disclosed herein include various methods of computer implemented virtual reality eating behavior training as described above. One representative method embodiment includes the following steps:
a) using a computing system to evaluate an input of one or more patient eating behaviors;
b) determining, with the computing system, a patient eating disorder based at least in part upon the input;
c) determining, with the computing system, a target eating behavior based, at least in part, upon the patient eating disorder;
d) generating, with the computing system, a virtual scenario for training the patient to accomplish the target eating behavior;
e) presenting the virtual scenario to the patient, through a virtual reality interface, where the virtual scenario allows the patient to interact virtually with the computing system to virtually accomplish the target eating behavior;
f) monitoring, through the virtual reality interface, one or more patient actions within the virtual scenario; and
g) providing real-time feedback to the patient, through the virtual reality interface, based upon the one or more patient actions within the virtual scenario, said feedback being configured to encourage the target eating behavior.

Other method embodiments may utilize at least one of artificial intelligence functionality or machine learning functionality to perform an evaluative step. Method embodiments may include generating and presenting a virtual scenario including a visual output, an audible output and at least one of a tactile output and an olfactory output. Methods may include detecting biofeedback from the patient while monitoring patient actions within the virtual scenario. The detected biofeedback may include eye motion, pupil dilation, blink rate, skin temperature, perspiration level, breathing rate, cardiac pulse rate, blood oxygen level and blood pressure or other biological parameters.

A method embodiment may include presenting one or more successive eating approximations configured to initiate the target eating behavior in the patient. Successive eating approximations can include are not limited to setting a table, pouring a drink, placing a food on a plate, picking up the food with a fork, and smelling the food. A virtual scenario may include a food selection scenario, configured to allow the patient to virtually select at least one item from the list of fear or forbidden foods for consumption.

Method embodiments may also include generating a virtual human guide configured to model the target eating behavior within the virtual scenario. A method embodiment may include presenting a virtual stimulus to elicit a reward response in the patient. The methods may also include determining whether the patient eating disorder is in remission.

In the following description, for the purposes of explanation, numerous specific details are set forth to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

### Specific Exemplary Embodiments

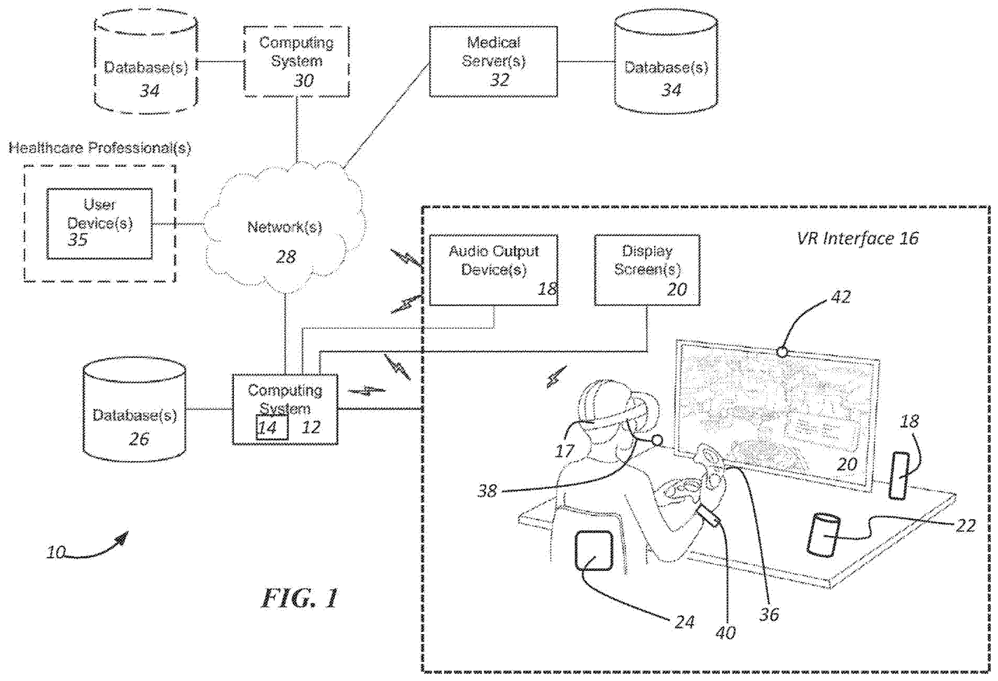

We now turn to the embodiments as illustrated by the drawings. FIG. 1 is a schematic block diagram of a representative VR eating behavior training system 10. The VR eating behavior training system 10 includes a computing system 12 having a processor 14 or being in communication with a processor 14 coupled to one or more VR devices that make up a VR interface 16. The VR interface 16 may worn or otherwise engaged with by a patient. The VR interface 16 may include one or more audio output devices 18, display screens 20, olfactory output devices 22, tactile output devices 24, and databases 26. The computing system 12 may further be coupled, via one or more communication networks 28, to a clinician's (e.g., a healthcare professional's) remote system 30, one or more medical servers 32 respectively coupled to one or more medical databases 34, and any number of other computing systems respectively coupled to one or more databases.

In various embodiments the computing system 12 or other computing systems may be implemented with, without limitation, desktop computers, single-board computers, tablet computers, laptop computers, handheld computers, edge devices, wearable devices, and the like, running an appropriate operating system. Thus, the computing system 12 may be a host machine to which the VR interface 16 is coupled. The host machine may be configured to generate data and signals presented to the patient via the VR interface 16, creating a VR environment through which the patient may be placed in various scenarios, and perform various training and exercises as described herein. Accordingly, in various embodiments, the VR interface 16 may include, without limitation, VR headsets 17, VR simulators, VR glasses, VR goggles, screens, monitors, projections, holograms, or other suitable devices for generating or otherwise visually displaying VR environments. In addition, the VR interface 16 may further include one or more augmented reality (AR) devices, such AR glasses, AR goggles, AR headsets, smartphones, or other screens and/or monitors. Thus, in some embodiments, the one or more VR interfaces 16 may be peripheral devices that communicate with the computing system 12 to display the VR interface. In other embodiments, the VR interface 16 may include all or part of the computing system, and the VR device itself may generate the VR environment that is displayed to a patient.

Healthcare professionals, such as a clinician, case manager, physician, or other medical provider, may be able to interact with the patient virtually in the various VR environments. Thus, a healthcare professional user device 35 may be coupled to the computing system 12 or communicate with the computing system 12 across one or more networks 28. Alternatively, the computing system 12 and user device 35 may be coupled to the one or more medical servers 32, which may be configured to host instances of the various VR environments presented to the patient and/or healthcare professional. Thus, the healthcare professional may communicate and/or interact with the patient virtually via the one or more medical servers 32 and other system elements. The one or more medical servers 32 may further be configured to monitor, track, and log the actions of the patient in the VR environment, or to monitor, track, and log other input to the VR eating behavior training system 10 as described herein. Inputs from the healthcare professional (e.g., notes from a clinician) may be saved along with patient actions or patient input in a local database 26, a remote database 34, and the data may be shared with one or more healthcare professionals via the one or more medical servers, 32 or transmitted directly to the respective healthcare user devices 35.

In yet further embodiments, additional users may similarly interface with the computing system 12, and/or medical servers 32 to interact with the patient virtually. The additional users may be coupled to the computing system 12 or medical servers 32 through their own computing systems.

As noted above, the VR interface 16 will typically present visual information to the patient using any number or combination of VR or AR headsets 17, simulators, glasses, goggles, screens, monitors, projections, holograms, and the like, which are capable of presenting information visually. In addition, a typical VR interface 16 will include speakers, headphones, earbuds, or similar apparatus for presenting audible information to the patient, with the visual and audible information together creating the virtual environment.

In some embodiments, the virtual environment can be supplemented or enhanced with olfactory information. Thus, the VR interface 16 may include one or more olfactory output devices 22. A non-limiting representative olfactory output device is described in U.S. patent application Ser. No. 16/837,456 (the “'456 Application”; U.S. Publication No. US 2020/0222658), filed Apr. 1, 2020, by Michael Leon, a co-inventor herein, entitled “Cognition and Memory Enhancement Via Multiple Odorant Stimulation.” The disclosure of the '456 application is incorporated herein by reference in its entirety for all purposes. The device disclosed in the '456 application can output 12 or more distinct odors, which can in certain embodiments described more fully below be food odors designed to enhance a virtual scenario.

Similarly, the virtual environment may optionally be supplemented or enhanced with tactile information provided through a tactile output device 24. Tactile information can be, but is not limited to, heating or cooling perceived by the patient, subsonic energy, pressure, or similar outputs that are felt by the patient. Representative tactile output devices 24 include, but are not limited to, heating pads or other heaters, cooling pads or other coolers, fans, mechanical massaging or kneading furniture, garments, or devices, pressure applying cuffs, clothing or furniture, subsonic speakers, ultrasonic speakers, and the like.

The computing system 12 can receive input from the patient through more handheld controllers 36, joysticks, a keypad, mouse, or similar electronic input devices. In addition, the VR device 16 may include a microphone 38. The microphone 38 may be a separate component in communication with the computing system or incorporated into the VR headset 17, display screen 20 or another component. The microphone 38 can be configured to receive verbal or other audible input for the patient. Patient interaction within the VR environment can be purposefully or intentionally communicated from the patient to the VR eating behavior training system 10 through input devices such as a handheld controller 36 or and microphone 38. Additional input may be collected by the VR eating behavior training system 10 autonomously. For example, the computing system 12 may be in communication with one or several detectors or sensors, in particular biofeedback detector(s) 40. Representative biofeedback detectors 40 include but are not limited to apparatus such as an eye motion detector, a pupil dilation detector, a blink rate detector, a skin temperature detector, a perspiration level detector, a breathing rate detector, a cardiac pulse rate detector, a blood oxygen level detector, a blood pressure detector, and similar detectors or sensors.

Certain detectors or biofeedback sensors 40 may be implemented with a patient facing camera 42 or other optical sensor implemented in or in conjunction with the VR headset 17 or similar apparatus. For example, patient eye motion, pupil dilation, and blink rate can be detected with the camera or similar optical sensor as the patient interacts with the virtual environment. Other detectors will require additional apparatus in communication with the computing system 12 including but not limited to thermometers, moisture level detectors, breathing rate and pulse rate detectors, pulse oximeter apparatus, blood pressure cuffs, and similar apparatus.

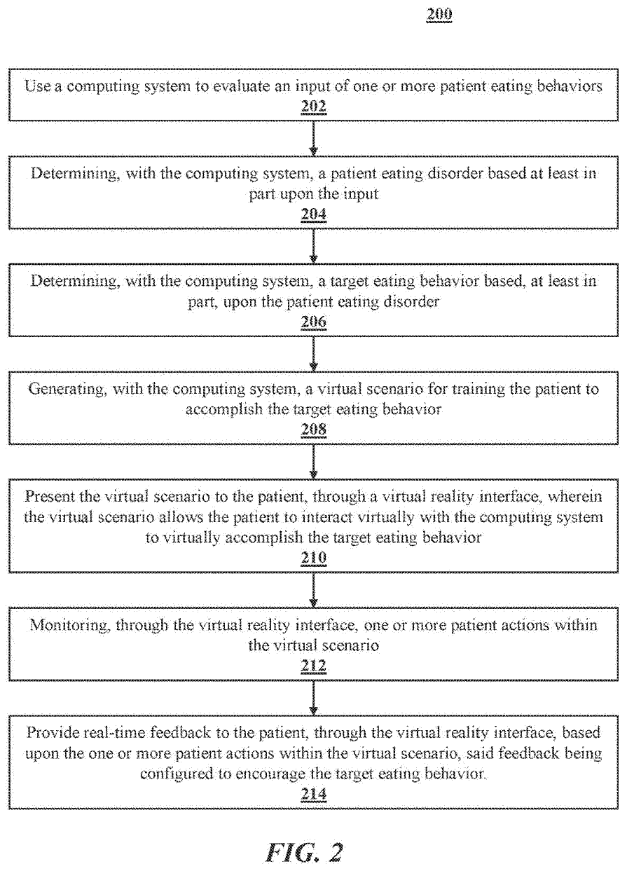

FIG. 2 is a flow diagram of a method 200 for VR eating behavior training, in accordance with various embodiments. The method 200 begins, at block 202, by evaluating, via a computing system, input representing at least one eating behavior of the patient. The eating behavior may be evaluated based upon input collected from the patient and processed with, for example, the computing system 12, a healthcare professional's remote system 30, or a medical server 32. Input from the patient may be collected directly through communication between a clinician and the patient, or through a computing system receiving written, verbal, or digital input. For example, input may be collected from the patient through the VR interface 16, and any associated handheld controller 36, microphone 38, biofeedback device 40 or other input device.

At block 204, the computing system determines at least one patient eating disorder based at least in part upon the input received in block 202. In various embodiments, an eating disorder may be evaluated based on one or more criteria for inward admission. Criteria for inward admission may include, without limitation, a body mass index≤13.5, a potassium level≤3.0 mmol/L, body temperature≤36° C., pulse≤38 beats per minute, binge-eating and vomiting several times a day and suicidal thoughts. Criteria for inward admission may, in some embodiments, be provided by a clinician, or self-reported by the patient.

At block 206, the method 200 may continue by determining a target or “normal” eating behavior for the patient. Determining a target or normal eating behavior may include determining a first threshold or goal for normal eating behavior. The first threshold or goal may include, for example, a pace or rate at which the patient consumes food, a portion or an amount of food consumed by the patient, food selection, avoidance of certain foods or demonstrating a willingness to eat other foods, eating in the presence of other people, avoidance of binge-eating, avoidance of purging, a satiety level, specifically, a rate at which the patient begins to feel full, or how full a patient feels after eating a given amount of food. Other suitable indicators of properly targeted eating behavior can be formulated to address, moderate, or treat the eating disorder determined in step 204.

At block 208, the method 200 further includes generating a virtual scenario with the computing system. The virtual scenario is constructed to incrementally train the patient to accomplish the target eating behavior. Thus, a virtual scenario may include many dozens or even hundreds of sessions where the patient interacts with system and progresses towards remission. As detailed herein, virtual scenarios may include different environments, social situations, or other simulations where the patient may practice the target eating behavior or develop advantageous eating and/or social habits in a VR environment. Virtual scenarios may include, without limitation, one or more successive eating approximations, e.g., VR approximations of the steps required to initiate eating. Eating approximations can be simple tasks performed in the virtual environment such as setting a table, pouring a drink, placing food on a plate, placing food on a fork, picking up food with a fork, smelling food, and finally eating the food.

Virtual scenarios may further include food selection scenarios, such as a virtual buffet or dessert cart from which appropriate food in appropriate quantities must be chosen, social scenarios in which one or more people are present, such as a birthday, or in some cases, situations in which an ancillary stimulus is presented. For example, ancillary stimuli in some virtual scenarios may include, without limitation, physical activities or exercises to be performed by a patient, exercise equipment, a staircase, or other stimuli that cause a reward response in the patient. In some embodiments, changes in virtual environment may further serve to remove certain cues or triggers that maintain the patient's abnormal eating behavior.

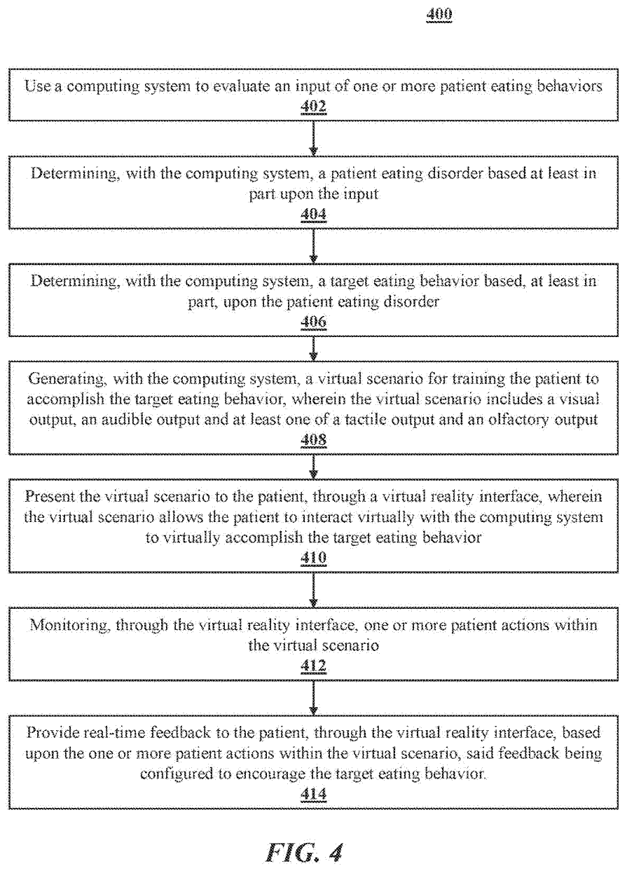

At block 210, the method 200 may continue by presenting the virtual scenario to the patient through the VR interface 16 and associated apparatus. As noted above, a representative interface may include, for example, a VR headset 17, an audio output device 18, an olfactory output device 22, and one or more tactile output devices 24. During the presentation of the virtual scenario to the patient, the method may include presenting a stimulus or a series of stimuli. As described more fully in association with FIG. 4 below, the stimulus may be visual, audible, scent-based, tactile or combination of stimuli. The stimulus, as discussed herein, may include, without limitation, any sort of food, beverage, social setting, physical activities or exercise to be performed by a patient, exercise equipment, a staircase, or other stimuli that causes a behavioral response in the patient affected by an eating disorder.

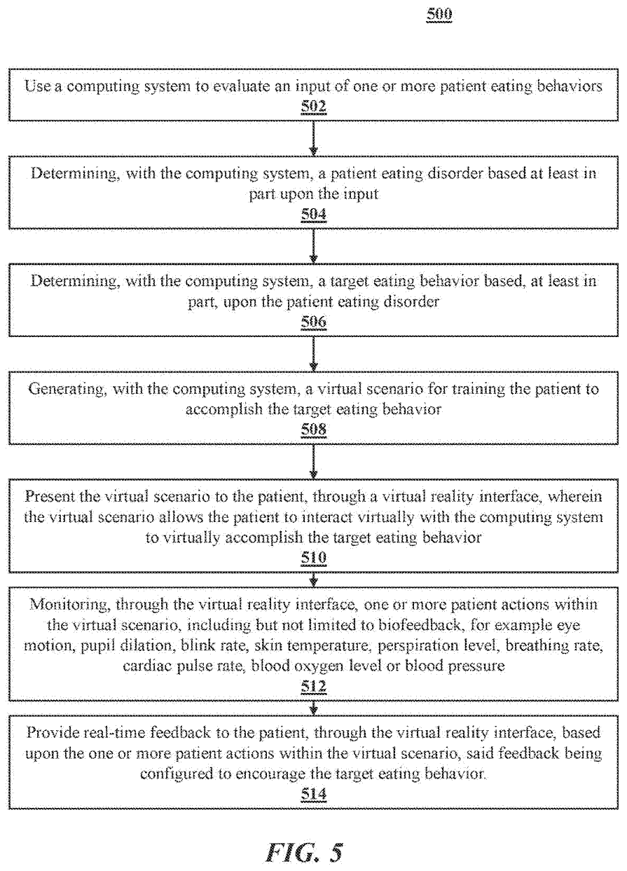

As a virtual scenario is presented to the patient the virtual scenario allows the patient to interact virtually with the computing system to virtually accomplish the target eating behavior, desirable social behaviors, and desirable physical behaviors. In addition, as noted at block 212, the system monitors, through the virtual reality interface, one or more patient actions within the virtual scenario. As noted above, patient actions may be purposefully input by the patient using, for example a handheld controller 36, microphone 38, keyboard, mouse, or other input device. Alternatively, a portion of the input from the patient may be collected with one or more biofeedback detectors as described more fully in conjunction with FIG. 5 below.

In various embodiments, the patient actions collected as the patient interacts with the virtual scenario may include food selection, a rate at which food is consumed, an amount of food that is consumed, a satiety level of the patient, the presence of one or more other individuals, whether the patient engages in binge-eating behavior, the way in which a physical activity is performed, such as a speed at which a patient ascends a staircase, a pace of walking and/or running, a duration of physical activity, etc. In some further embodiments, one or more patient actions may further include one or more physiological parameters of the patient during the virtual scenario typically collected with a biofeedback detector 40 or similar apparatus. Physiological parameters can be collected autonomously, and include but are not limited to, changes in heart rate or blood pressure, changes in skin salinity. In yet further embodiments, the one or more patient actions may further include emotional responses of the patient. Emotional responses may include measurements of skin salinity, audio recordings for vocalizations, eye movements, or self-reporting of emotional state by the patient. Thus, patient actions may include any actions taken by the patient within the virtual scenario, and further may include recorded or autonomously sensed physiological and emotional responses of the patient to the virtual scenario.

At block 214, the method 200 may continue by providing real-time feedback to the patient through the VR interface, typically while in the virtual scenario. Providing feedback may include providing instruction, audibly or visually within the virtual scenario. Feedback may be provided by a virtual avatar or the representation of a human being, through demonstration, text instructions, or picture instructions, or through a combination of different types of instruction as described above. For example, in some embodiments, a clinician may be present with the patient virtually, within the VR scenario. In other embodiments, a model guide may be provided to act as a reference for food selection and other normal eating behavior. In yet further embodiments, providing feedback may include reporting results and/or showing real-time measurements of pace of food consumption, amount of food consumption, physiological parameters, or other feedback through which a patient may monitor their actions and progress in real-time. In some embodiments, feedback may be tied to a threshold or interim goal as described above. Thus, feedback may be configured to instruct the patient to meet the first or a successive threshold for normal eating behavior or otherwise accomplish or encourage the target eating behavior.

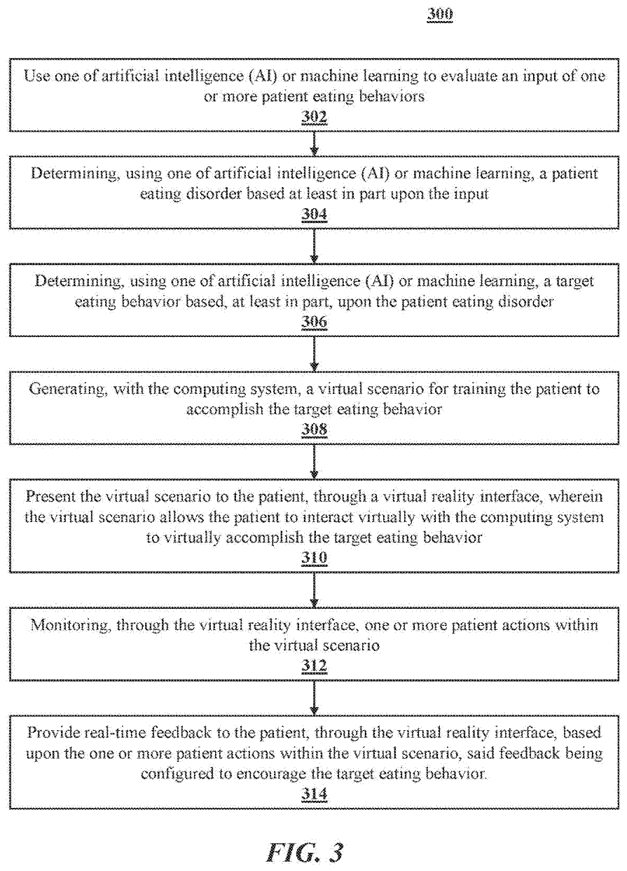

FIGS. 3-7 are flow diagrams of certain alternative methods 300, 400, 500, 600, and 700 for VR eating behavior training. The alternative methods illustrated in FIGS. 3-6 are non-limiting, representative examples. Each of the alternative methods is implemented with a computing system and aspects of the method are presented to a patient through a VR interface.

In the FIG. 3 embodiment, the method 300 at block 302 includes the use of artificial intelligence (AI) and/or machine learning to evaluate the input of one or more patient eating behaviors. In addition, AI or machine learning may be used for other steps where a decision or evaluation is made by a computing system. For example, at block 304, the method 300 includes using AI and/or machine learning to determine the patient eating disorder based at least in part upon the patient input. Alternatively, AI or machine learning may be used to determine the target eating behavior based, at least in part upon the patient eating disorder (block 306). Steps 308, 310, 312, and 314 correspond to steps 208, 210, 212 and 214 described in detail above. The use of AI or machine learning functionality can in certain instances advantageously limit the involvement of a clinician or other healthcare professional and overall treatment program. Thus, AI or machine learning provides a computing system with the capacity to analyze input, develop, and present a specific therapeutic virtual environment for eating behavior training with little or no input from healthcare professionals or outside clinicians as the treatment program progresses.

The FIG. 4 embodiment illustrating method 400 includes steps 402, 404, 406, 410, 412, and 414, which correspond to steps 202, 204, 206, 210, 212, and 214 described in detail above. Block 408 of method 400 involves generating with the computer system a virtual scenario for training the patient to accomplish the target eating behavior, including a visual output, an audible output and at least one of a tactile output and an olfactory output. Olfactory output may be provided through an olfactory output device 22. A representative olfactory output device 22 is described in the '456 Application referenced above. The device disclosed in the '456 application can output 12 or more distinct odors, which in certain embodiments can be food odors configured to enrich the virtual scenario and provide the system with additional opportunities to train the target eating behaviors.

Similarly, the virtual environment may be supplemented or enhanced with tactile information delivered through the tactile output device 24. Tactile information can be, but is not limited to, heating or cooling perceived by the patient, subsonic energy, pressure, therapeutic massage, or similar tactile outputs that are felt by the patient. Representative tactile output devices 24 include but are not limited to heating pads or other heaters, cooling pads or other coolers, fans, mechanical massaging or kneading apparatus, garments, furniture or other devices, pressure applying cuffs, clothing or furniture, subsonic speakers, ultrasonic speakers, and the like.

The method 500 of FIG. 5 includes enhanced monitoring and feedback functionality. Thus, steps 502, 504, 506, 508, 510, and 514 correspond to steps 202, 204, 206, 208, 210, and 214 described in detail above. Block 512 describes enhanced monitoring of patient actions through the VR interface, by collecting data through one or more biofeedback detectors 40. Representative biofeedback detectors 40 include but are not limited to apparatus such as an eye motion detector, a pupil dilation detector, a blink rate detector, a skin temperature detector, a perspiration level detector, a breathing rate detector, a cardiac pulse rate detector, a blood oxygen level detector, and a blood pressure detector.

Certain detectors or biofeedback sensors 40 may be implemented with a patient facing camera 42 or other optical sensor implemented in or in conjunction with the VR headset 17 or similar apparatus. For example, patient eye motion, pupil dilation, and blink rate can be detected with the camera or similar optical sensor as the patient interacts with the virtual environment. Other detectors will require additional apparatus in communication with the computing system 12 including but not limited to thermometers, moisture level detectors, breathing rate and pulse rate detectors, pulse oximeter apparatus, blood pressure cuffs, and similar apparatus.

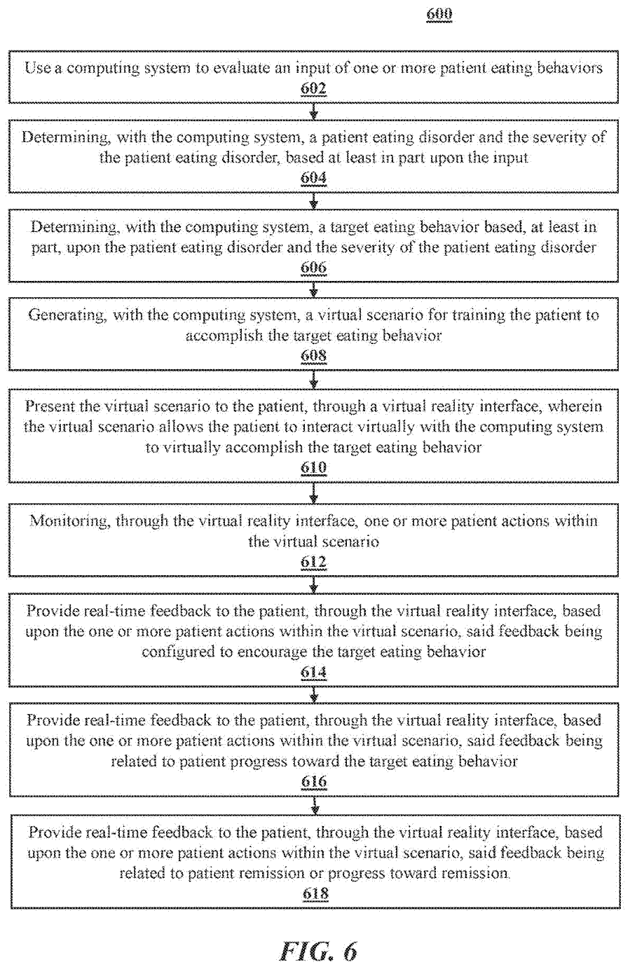

The method 600 of FIG. 6 includes additional or supplemental analysis performed by the computing system, possibly using AI or machine learning to process input collected from the patient within the virtual scenario. For example, the method 600 includes a determination by the computing system of the severity of the patient eating disorder. The system determines the target eating behavior based at least in part upon the severity of the patient eating disorder (steps 604 and 606). The feedback provided to the patient through the VR interface over time in successive sessions may then include real-time feedback concerning progress toward the target eating behavior or goals and/or real-time feedback concerning progress towards remission. Thus, the method 600 includes using the computing system to evaluate the patient's progress toward and distance from certain eating targets, goals, or remission. This functionality may be implemented using AI or machine learning. Machine implemented evaluation of progress towards goals or mission can serve to limit or eliminate the necessity of human support while the patient utilizes the VR eating behavior training system 10. Blocks 602, 608, 610, 612, and 614 are like the corresponding steps of method 200.

Determining that a patient is in remission may include determining whether the patient meets one or more remission criteria. Thus, remission criteria may include the consumption of food at a suitable rate, for example 350 g in 12-15 minutes. Other remission criteria may be demonstrated willingness to eat fear foods or forbidden foods, or consuming a portion of food below a threshold limit.

In some embodiments, the system may test for remission periodically. In some examples, the patient may subsequently be tested, virtually, to confirm the patient remains in remission. In some embodiments, remission testing may be conducted every 6 weeks. Remission testing may be used by the patient to boost the patient's confidence virtual situations, before certain real-world activities, for example before a patient encounters or enters a difficult life situation. Furthermore, remission testing may detect and/or predict patient relapse. In some embodiments, if relapse is detected, the patient may be re-evaluated for an eating disorder, and re-admitted for treatment and further eating behavior training.

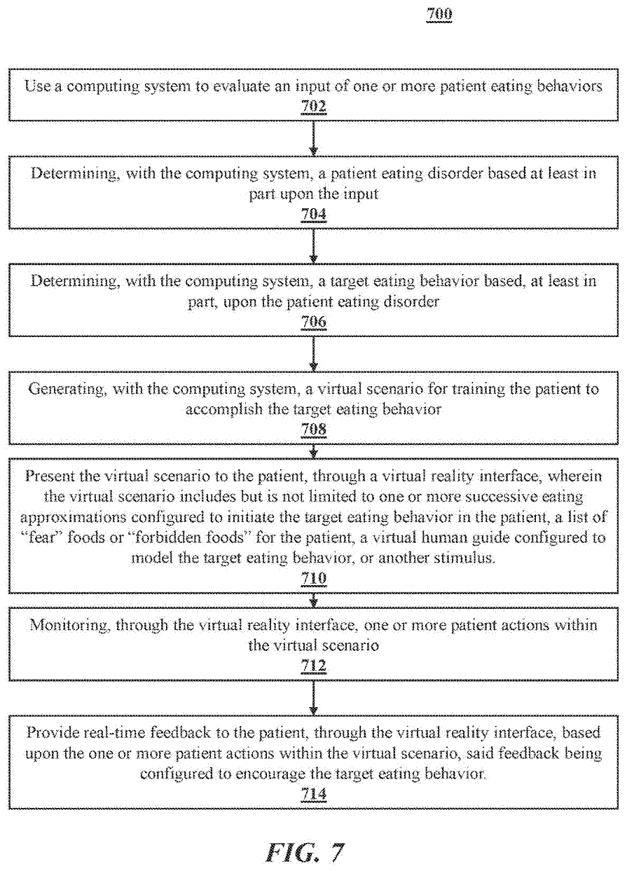

The method 700 of FIG. 7 includes the presentation of a virtual scenario through the VR interface 16 presenting, but not limited to, one or more successive eating approximations configured to initiate the target eating behavior in the patient. Alternatively, the virtual scenario of method 700 may include use of a list of “fear” foods or “forbidden foods” for the patient. The virtual scenario may include a virtual human guide configured to model the target eating behavior, or another feature or stimulus. Steps 702, 704, 706, 708, 712, and 714 of the method 700 are substantially the same as the corresponding steps shown in method 200.

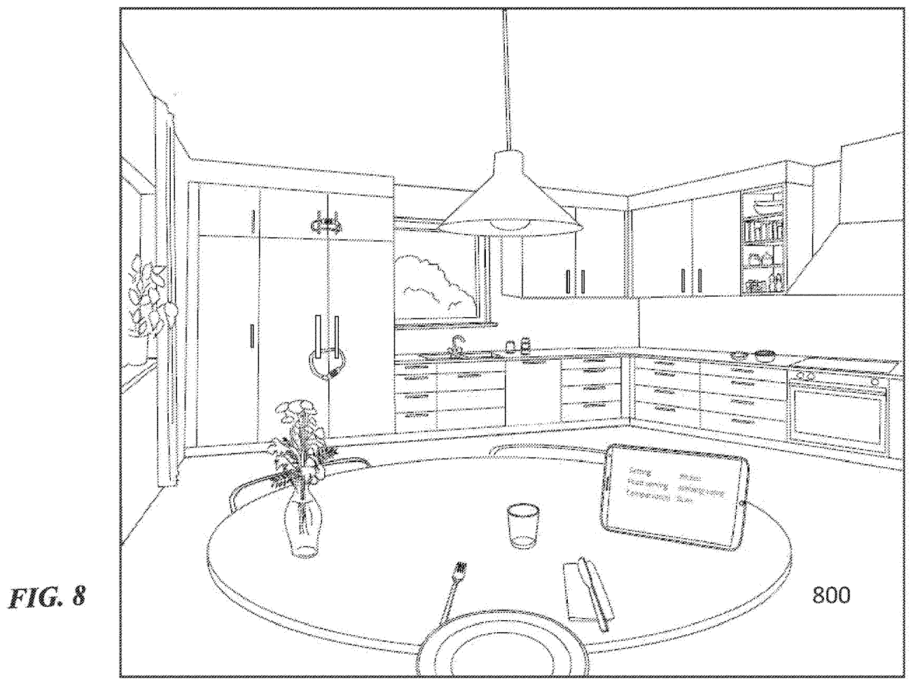

Additional methods are described below with reference to FIGS. 8-20. FIGS. 8-20 are representative non-limiting examples of VR device images such as may be presented to a patient through an eating behavior training system 10. It is important to note that the renditions of FIGS. 8-20 could be presented to a patient as still images, but it is far more likely that similar images would be presented as video data, including but not limited to three-dimensional video data with optional sound, scent-based, and/or tactile output, all configured to create a realistic and engaging virtual scenario. The VR scenarios described with respect to FIGS. 8-20 are generated by a computing system based upon input of one or more patient eating behaviors, as described in method 200 steps 202, 204, 206, and 208. See also the corresponding steps of methods 300, 400, 500, 600 and 700.

FIGS. 8, 9, and 10 illustrate a series of successive VR approximations for initiating eating behavior when patients do not eat or drink normally, and/or engage in binge-eating. The successive scenarios presented via the VR interface 16 permit a patient to practice how to initiate a meal, how to eat a meal, and otherwise engage in or emulate a target eating behavior. Over a period of time, the VR interface 16 may be used to present the successive VR approximations to a patient, as will be described in greater detail below.

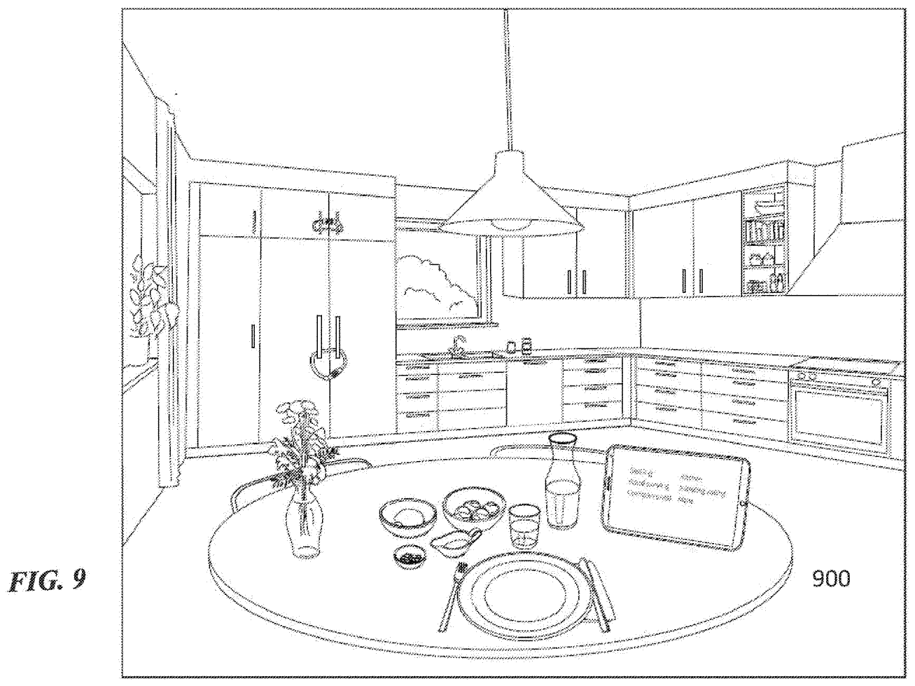

FIG. 8 illustrates a VR interface 16 rendition 800 of a table setting created for the treatment of anorexic behavior. In this embodiment, the VR eating behavior training system 10 may be configured to present, via the VR interface 16, a scenario in which the patient is asked to virtually set a table for a meal. In some scenarios, the patient may be assured that they do not have to eat during the exercise. FIG. 9 illustrates a subsequent VR rendition 900 of a serving of water and condiments. The rendition 900 is also useful for treatment of anorexic behavior. At this stage, the VR system 10 may be configured to present a scenario in which the patient is asked to pour water into the glass. As above, the patient may be assured that they do not have to drink what is poured into the glass.

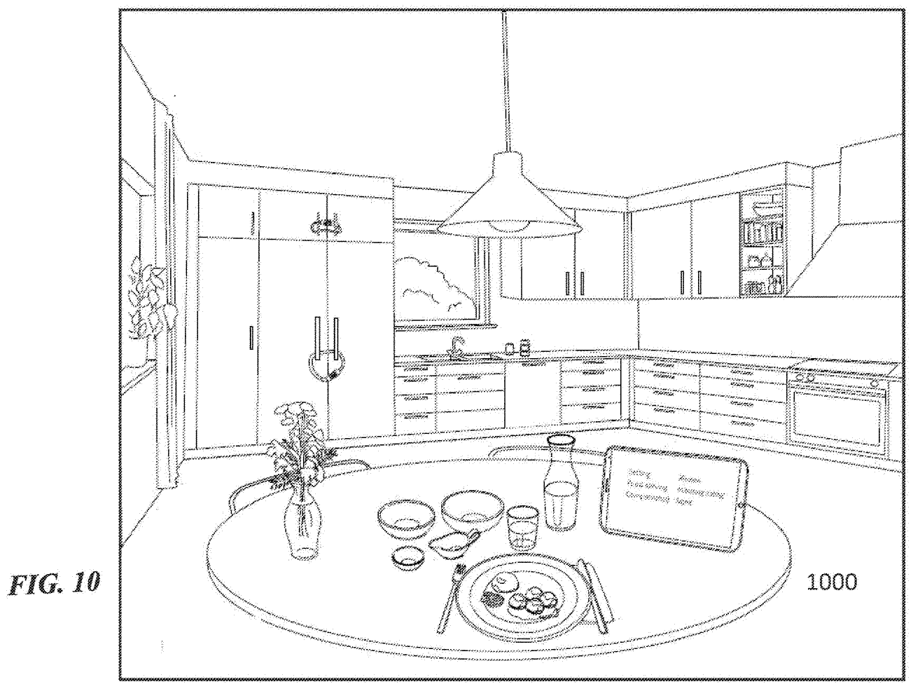

FIG. 10 illustrates a VR rendition 1000 of a serving of food on a plate, again for the treatment of anorexic behavior. In this later scenario, the patient may be prompted to perform further exercises. For example, the patient may be asked to put food on the plate. The patient may be assured that they do not have to eat the food. The patient may further be prompted to cut the food into pieces, pick up food with a fork, or to smell the food. This iterative process may continue through multiple sessions and several virtual scenarios until the patient puts food into their mouth and eating is initiated.

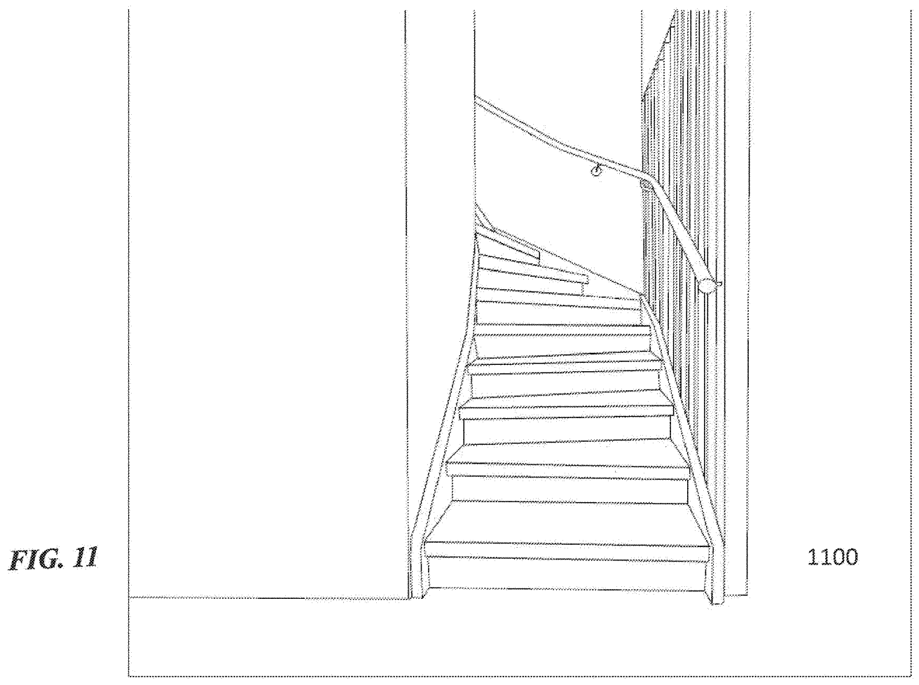

In some embodiments, the VR interface 16 can present a virtual reality that does not contain food or beverage but is instead associated with the treatment of other symptoms of an eating disorder. For example, as illustrated in FIG. 11, the VR system 10 may be configured to present a rendition 1100 of a staircase in a virtual environment as a non-rewarding stimulus in the treatment of anorexic behavior. Presentations of anorexic behavior may include a patient that eats too little food such that their caloric intake that is below what is generally considered a healthy level. Alternatively, anorexic behavior may involve a patient who engages in excessive physical activity. In such patients, when the patient eats too little food, or engages in an excess physical activity, the brain's systems for reward and attention are activated. Thus, for these patients, both dieting and high levels of physical activity are initially rewarding, and subsequent anorexic behavior is maintained through conditioning to the stimuli that originally provided the reward.

Therefore, in the FIG. 11 embodiment, the VR interface 16 may be configured to present a virtual rendition 1100 of a non-food or eating stimulus to trigger the reward systems of the patient's brain. In the example depicted, a staircase may be presented as a stimulus that will maintain the anorexic behavior (e.g., remind and reward the patient of the feeling of losing weight), since a patient who is running up and down a physical staircase at home or work may feel rewarded as they are engaged in excessive physical activity. Thus, the VR interface 16 may be used to present a virtualized experience of the stimulus that produces a reaction without the negative effects of anorexic behavior. The patient may thus, in some embodiments, be trained via the VR interface 16 to walk slowly up the staircase (as opposed to running). After effective treatment, when the patient has learned how to eat and how to maintain healthy eating and exercise habits, a staircase may no longer trigger feelings of reward.

The VR eating behavior training system 10 may be used to treat any sort of eating disorder. For example, a system 10 may be used to train a patient who is unable to eat in the presence of others. Virtual reality is advantageous for this sort of training because the patient can physically be alone, but virtually in the presence of a virtualized clinician, virtual party guests or other people as described in detail below. The patient is cognizant of the differences between virtual reality and actual interaction with other persons, therefore a virtual environment may be easier to function and make progress within for many patients. A virtual reality is not limited by real-world constraints. For example, the VR environment may initially present an automatically locking refrigerator once a food selection has been made. The locked refrigerator may virtually restrict access to food in the VR environment, thus preventing the patient from engaging in virtual binge-eating behavior. The automatic locking feature of the virtual refrigerator can be modified after progress has been made toward a healthy eating target.

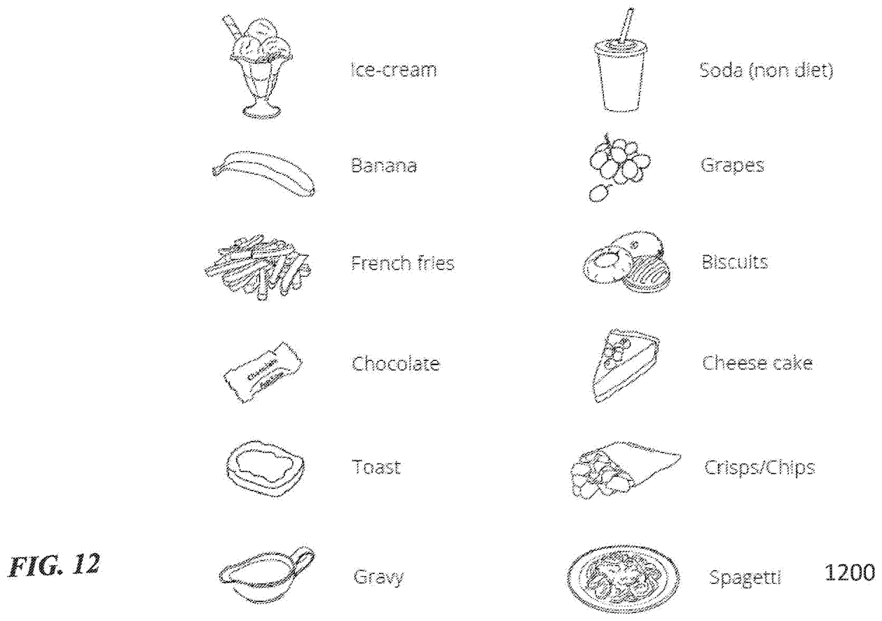

FIG. 12 illustrates a rendition 1200 of a list of “forbidden foods” or “fear foods.” Patients with eating disorders often are fearful of eating or even tasting any number of forbidden foods or fear foods. Thus, a virtual presentation of a specific patients forbidden foods or fear foods can be utilized in the VR treatment of eating disorders. Initially, a patient may be able to provide a list of forbidden foods or fear foods applicable to themselves through a VR interface 16. In some embodiments, the VR interface may be configured to prompt the patient to list one or more forbidden foods. The VR system may then generate a list of forbidden foods based on a series of patient responses. Rendition 1200 is a representative list of forbidden foods generated in VR by the patient. Subsequently, the system 10 may generate and present VR scenarios featuring the forbidden foods or fear foods. Thus the, the system 10 assures that each VR scenario created is specifically tailored to the needs and requirements of a given patient.

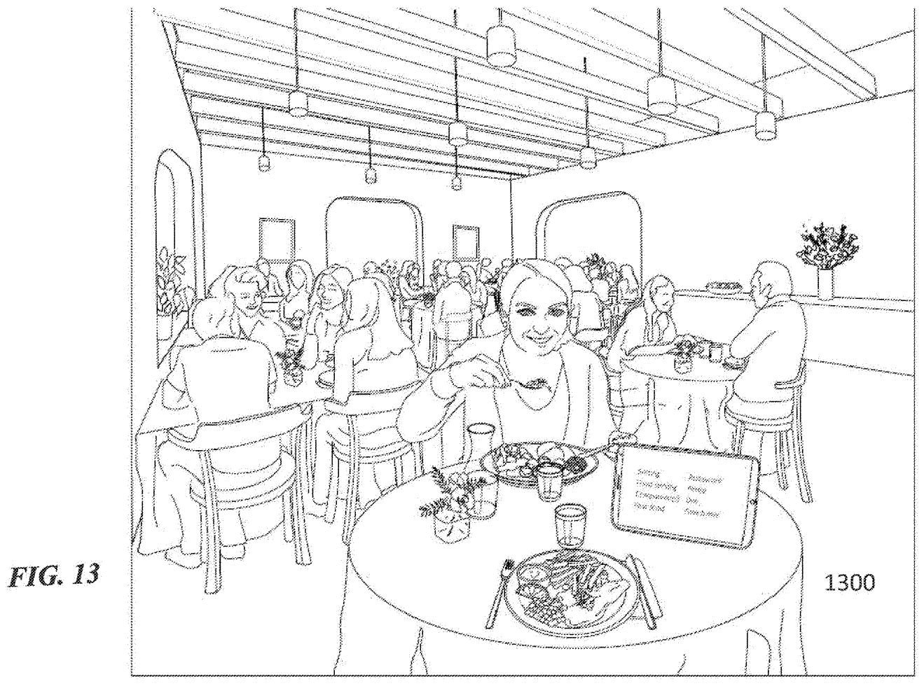

FIGS. 13-18 illustrate a series of representative VR training scenarios such as could be used to train a patient to consume forbidden or fear foods, or to consume foods in a restaurant, social setting or other difficult situation. Training can take place at a virtual clinic, a virtual café restaurant, or at a virtual representation of the patient's home. The system 10 can generate a series of progressively more difficult VR scenarios suitable for a given patient. For example, a relatively easy, although still difficult, scenario for some patients is eating at home. On the contrary, it is often a very difficult scenario for many patients to eat in public where a broad range of food choices must be made, for example at a buffet or smorgasbord. Therefore, when patients initiate treatment, there are often only few food options offered to them. These food options may be increased as patients progress in VR treatment. Selection of food is also difficult for bulimics, who want to “eat it all.” When such patients have taken what they consider to be one bite of food too many, they may rapidly conclude that they may as well continue eating everything they see.

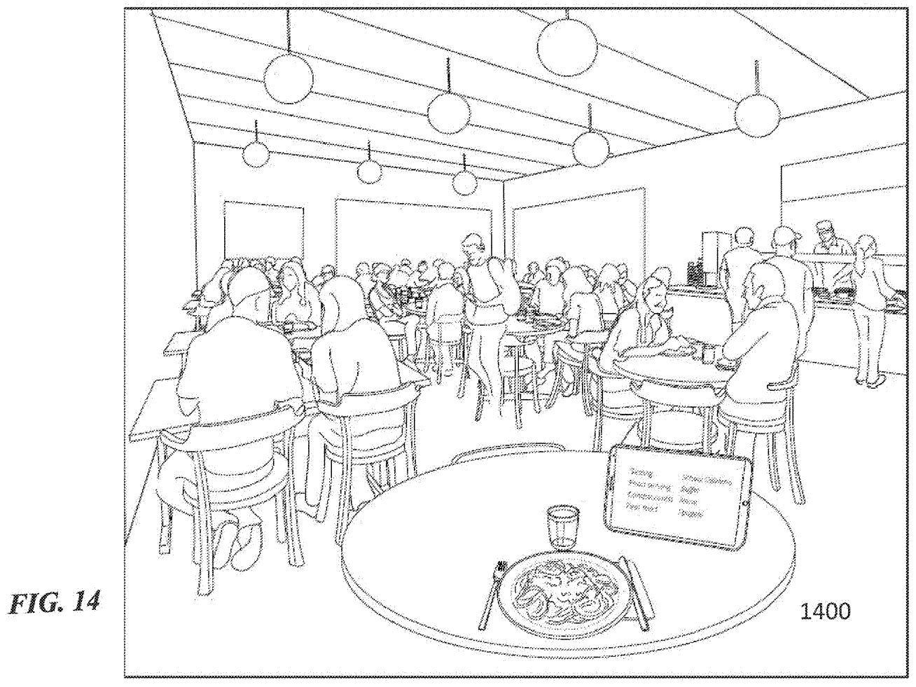

These and other symptoms or behavioral attributes of an eating disorder may be treated by the system 10 with appropriately designed virtual scenarios. For example, FIG. 13 illustrates a VR rendition 1300 of a virtual guide assisted “forbidden food” training scenario. As depicted, the forbidden food may be french fries. The patient may be trained to eat the forbidden food, virtually, in the presence of a virtual guide, clinician or other mentor. FIG. 14 is a VR scenario rendition 1400 presenting an unassisted “forbidden food” training scenario. In this virtual environment, the patient may practice eating the forbidden food, in this example spaghetti, unassisted by a virtual clinician or helper. The system 10 can be configured to present either assisted or unassisted scenarios during a course of treatment as feedback from the patient is processed and as they progress toward their target eating goals.

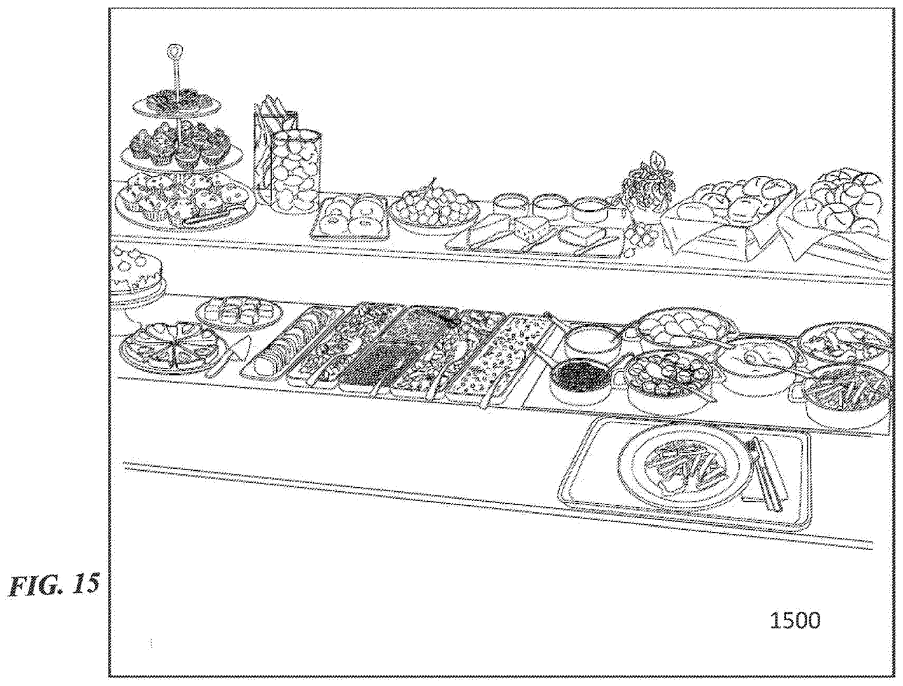

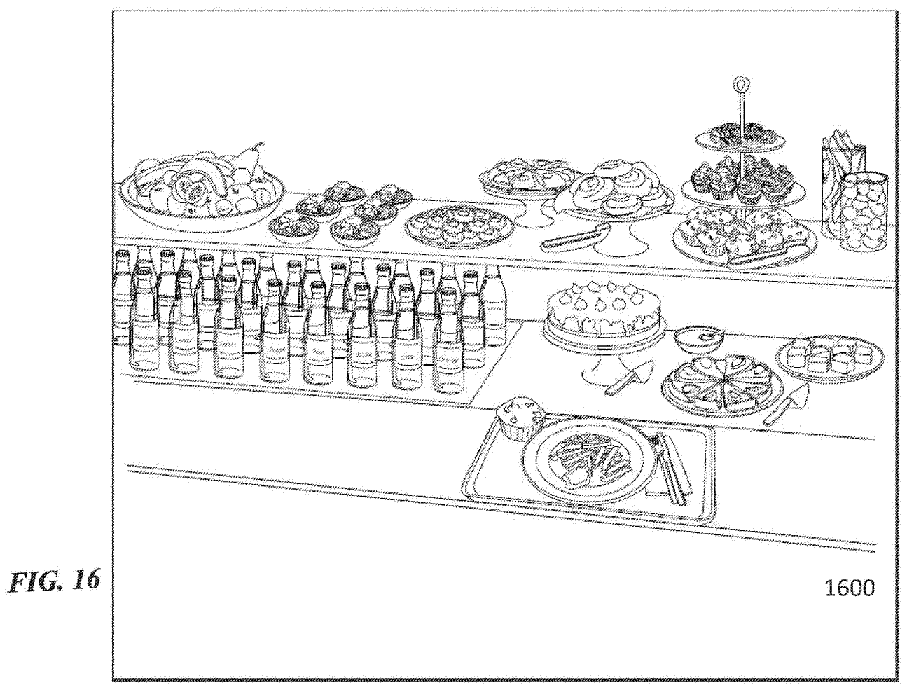

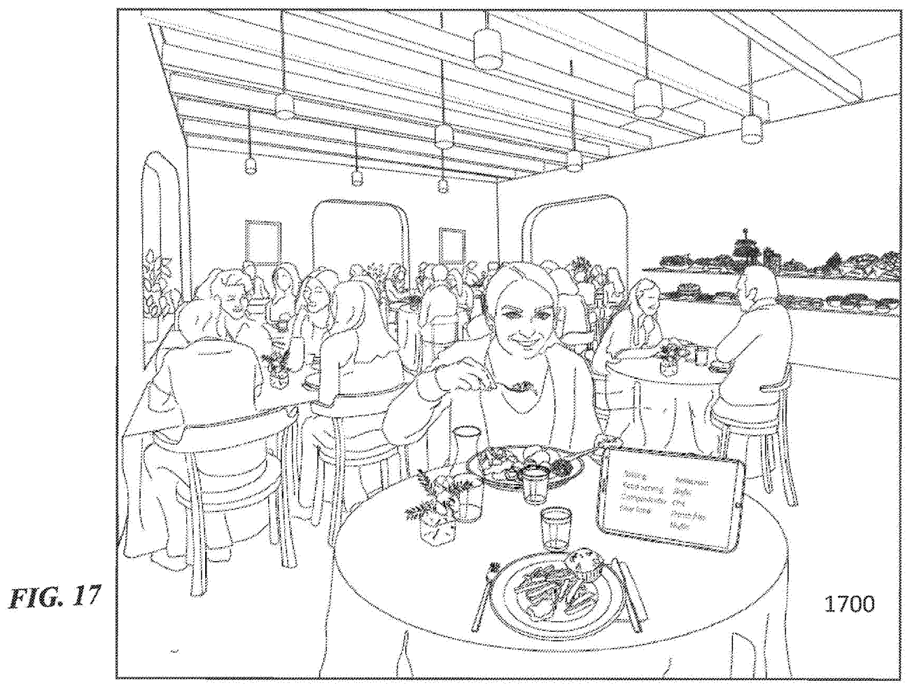

FIGS. 15-17 illustrate various alternative food selection training scenarios 1500, 1600, and 1700. FIG. 15 illustrates a VR interface 16 presenting a food selection training scenario 1500 where the patient may practice selecting various foods from a buffet-style spread of food and drinks. Moreover, the buffet-style spread may include one or more forbidden foods, which the patient may practice selecting for their meals. FIG. 16 represents a virtual dessert cart training scenario 1600. In this scenario, a patient practices the selection of desserts from the large choice available. FIG. 17 illustrates a VR interface 16 presenting a food selection scenario 1700 using two “forbidden foods” with a virtual model guide. In this embodiment, the patient may practice and/or test eating two forbidden foods, for example french fries and a muffin.

In some embodiments, any model guide may be a virtual avatar of another individual, such as the clinician, a friend or family member, or another role model. In other embodiments, the model guide may be a virtual entity not associated with a human user. The model guide may act as a guide, reference, or companion for the patient to encourage eating the forbidden food, or otherwise encourage the patient to engage in a target eating behavior or to avoid destructive eating behavior. Accordingly, the model guide may guide, provide a reference. or simply be an example indicating a desired portion or pace at which to consume food. Thus, the patient may, in some embodiments, copy, mirror, or otherwise approximate the actions of the model guide with respect to the consumption of food including forbidden foods.

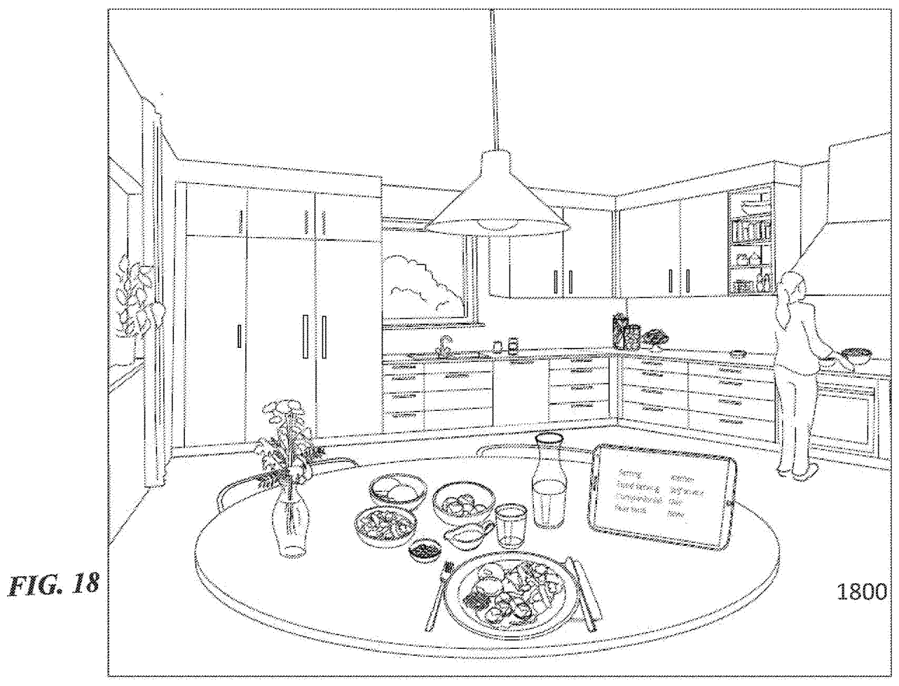

FIG. 18 illustrates a VR interface 16 presenting a normal eating behavior scenario 1800. Once the patient has practiced and achieved a target or normal eating behavior, the patient may need to continue to practice in a virtual environment to maintain the normal eating behavior. In scenario 1800, another individual may be present in the room with the patient, and no forbidden foods or fear foods may be presented. In some embodiments, the refrigerator in the VR environment may be unlocked, as the patient has been trained such that food is no longer a trigger for binge-eating.

In some VR scenarios, the patient may be encouraged to take risks. By setting up social goals and achieving these goals virtually, the patient's self-esteem will increase within the relatively non-threatening, but still realistic, virtual environment. For example, a scenario may include a schedule of challenges to be met by the patient periodically, for example every day. If the patient has managed her/his challenges for a period of time, perhaps one week, she or he is rewarded with a present. The schedule and rewards may be updated periodically, for example every week. The virtual scenario may be constructed such that only desired or targeted behavioral changes are rewarded.

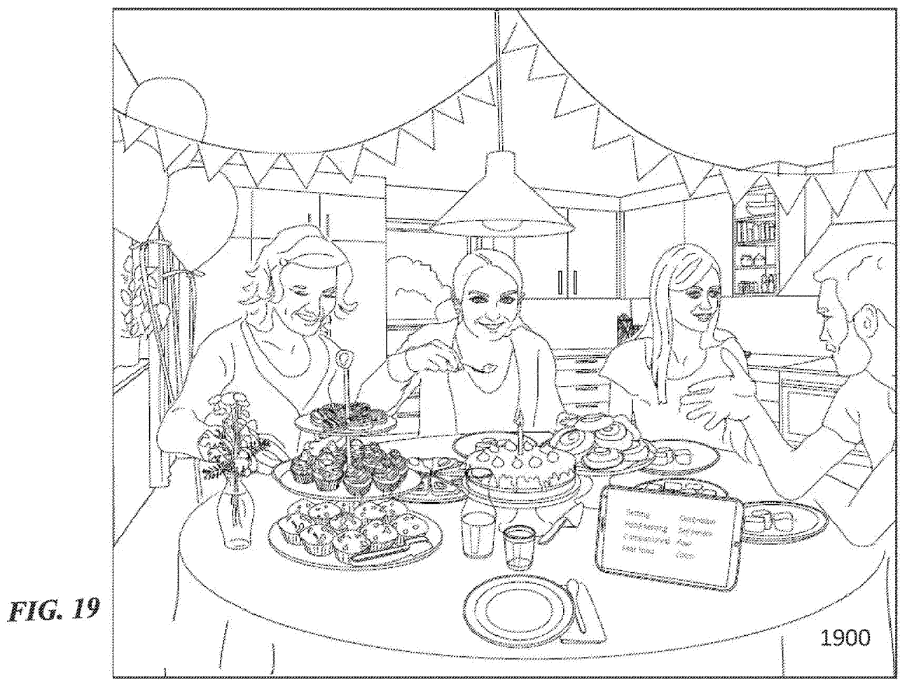

FIG. 19 illustrates a virtual risk-taking scenario 1900. In this embodiment, the risk-taking scenario is a birthday party. The system 10 may be configured to create virtual scenarios including any number of risk factors or risk scenarios based on the input from a given patient. In this example, a birthday party is an environment in which there are multiple other people, as well as food and drinks that the patient is expected to consume in an appropriate manner. Thus, the social setting tolerates neither abstaining, nor binge-eating, but expects normal social eating. In this scenario 1900, the patient may virtually practice entering a socially uncomfortable situation, while further practicing their ability to maintain normal eating behavior while in a troubling or socially high-risk scenario.

A patient may be less motivated to engage in abnormal eating behavior or abnormally limited or excessive physical activity if he or she is kept satiated throughout the day through a virtual interface. For example, when a patient virtually engages in physical activity deemed by the system tend to be excessive, the patient may be interrupted from the activity at random intervals. Eventually, the patient's excessive physical activity will be extinguished and replaced by other activities. Similarly, patients who engage in too little physical activity may be encouraged to exercise in a properly formulated virtual environment. Thus, in some embodiments, the VR system 10 may be configured to provide selection and tracking of physical activities, as well as social and eating activities, through carefully targeted virtual scenarios constructed by a computing system.

An eating disorder patient is often withdrawn from normal social interaction and is therefore isolated because of her or his illness. Virtual environments as described herein facilitate the gradual restoration of normal social relationships. Social interactions may be replicated and practice in the VR environment, for example the birthday party as described above with respect to FIG. 19. Virtual practice of difficult social situations may be the only socialization possible for patients with relatively extreme eating disorders. Treatment with the virtual reality system 10 may continue until a patient has trained themselves to be sufficiently comfortable to engage in real-world socialization.

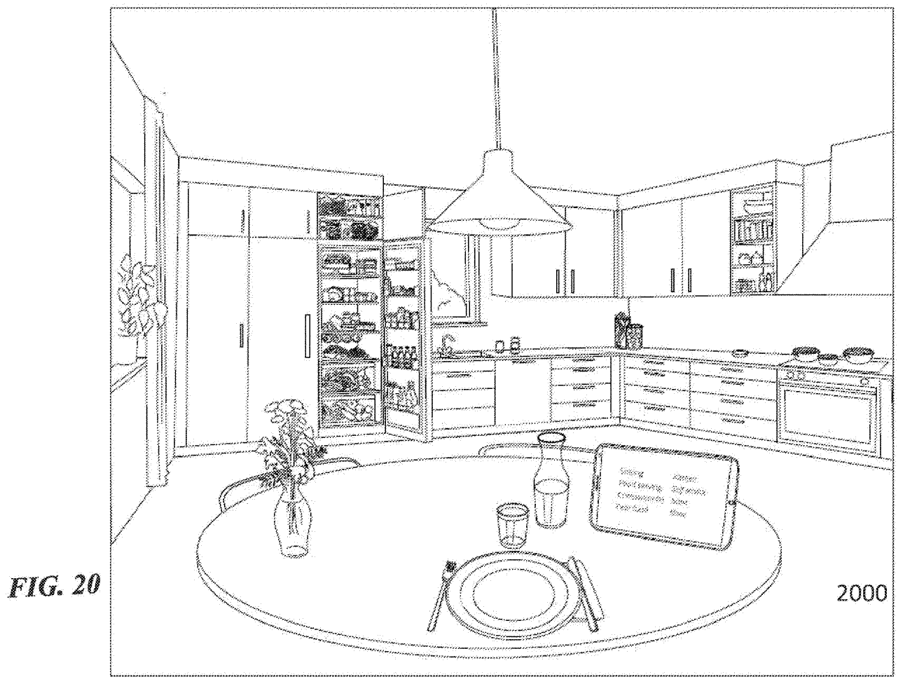

FIG. 20 illustrates a VR interface including a testing scenario 2000 for remission confirmation. In this embodiment, once the patient has overcome their eating disorder, or has made progress towards overcoming their eating disorder, and has therefore demonstrated the ability to maintain normal eating behavior, the patient may be tested periodically in a virtual environment for remission. For example, if the patient demonstrates that they can eat at a target rate of 350 g of food in 12-15 minutes, and if the patient has no fear foods or forbidden foods, eating restrictions may be removed or modified by the system 10. In some examples, eating behavior may be tested virtually, via the VR system, every six weeks. To test for remission, the patient may be monitored for one or more remission criteria. In addition, in some embodiments, the patient's real eating behavior may be clinically monitored in conjunction with their virtual eating behavior in the VR environment. Remission criteria may include, without limitation, one or more of normal eating behavior and satiety (e.g., normal eating pace and portion size selection), normal body weight, normal psychiatric profile, normal blood tests, a return to school or work, engaging in recreational activities, and patient confirmation that food and weight are no longer regarded as problems. For bulimia, remission criteria may further include no binge-eating or purging in the last three months. Other eating disorders will have appropriate criteria for remission.

In summary, the VR eating behavior training system 10 can autonomously, or at least partially autonomously, design and present a series of therapeutic VR simulations based upon continuous input from the patient. The simulations can be specifically tailored to include a patient's fear or forbidden foods, problematic social situations, and physical behaviors associated with the patient's eating disorder. The simulations may progress over time as the patient achieves interim goals as detected by the system. The VR eating behavior training system 10 can operate at least partially independent of professional supervision. Furthermore, many patients will make relatively rapid and in-depth progress towards their target eating behaviors or other goals using a VR eating behavior training system 10, since the system can be utilized daily in the patient's own home. Each of the VR methods and systems described herein can and typically will be supervised by a healthcare professional at some level. In particular, virtual training as described herein to overcome an eating disorder may be advantageously supplemented with real-world eating, socialization and exercise practices.

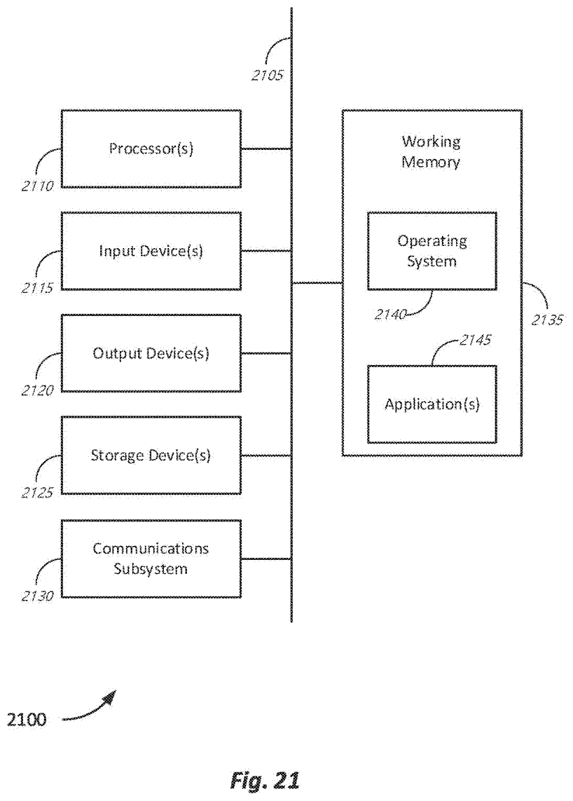

FIG. 21 is a schematic block diagram of a computer system 2100 for VR eating behavior training, in accordance with the various embodiments disclosed herein. The computer system 2100 is a schematic illustration of a computer system (physical and/or virtual), such as a control module, in-line controller, or host machine, which may perform the methods provided by various other embodiments, as described herein. It should be noted that FIG. 21 only provides a generalized illustration of various components, of which one or more of each may be utilized as appropriate. FIG. 21, therefore, broadly illustrates how individual system elements may be implemented in a relatively separated or relatively more integrated manner.

The computer system 2100 includes multiple hardware (or virtualized) elements that may be electrically coupled via a bus 2105 (or may otherwise be in communication, as appropriate). The hardware elements may include one or more processors 2110, including, without limitation, one or more general-purpose processors and/or one or more special-purpose processors (such as microprocessors, digital signal processing chips, graphics acceleration processors, and microcontrollers); one or more input devices 2115, which include, without limitation, a mouse, a keyboard, one or more sensors, and/or the like; and one or more output devices 2120, which can include, without limitation, a display device, and/or the like.

The computer system 2100 may further include (and/or be in communication with) one or more storage devices 2125, which can comprise, without limitation, local and/or network accessible storage, and/or can include, without limitation, a disk drive, a drive array, an optical storage device, solid-state storage device such as a random-access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer system 2100 may also include a communications subsystem 2130, which may include, without limitation, a modem, a network card (wireless or wired), an IR communication device, a wireless communication device and/or chipset (such as a Bluetooth™ device, an 802.11 device, a WiFi device, a WiMax device, a WWAN device, a low-power (LP) wireless device, a Z-Wave device, a ZigBee device, cellular communication facilities, etc.). The communications subsystem 2130 may permit data to be exchanged with a network (such as the network described below, to name one example), with other computer or hardware systems, between data centers or different cloud platforms, and/or with any other devices described herein. In many embodiments, the computer system 2100 further comprises a working memory 2135, which can include a RAM or ROM device, as described above.

The computer system 2100 also may comprise software elements, shown as being currently located within the working memory 2135, including an operating system 2140, device drivers, executable libraries, and/or other code, such as one or more application programs 2145, which may comprise computer programs provided by various embodiments, and/or may be designed to implement methods, and/or configured systems, provided by other embodiments, as described herein. Merely by way of example, one or more procedures described with respect to the method(s) discussed above may be implemented as code and/or instructions executable by a computer (and/or a processor within a computer); in an aspect, then, such code and/or instructions can be used to configure. and/or adapt a general-purpose computer (or other device) to perform one or more operations in accordance with the described methods.

A set of these instructions and/or code may be encoded and/or stored on a non-transitory computer readable storage medium, such as the storage device(s) 2125 described above. In some cases, the storage medium may be incorporated within a computer system, such as the system 2100. In other embodiments, the storage medium may be separate from a computer system (i.e., a removable medium, such as a compact disc, etc.), and/or provided in an installation package, such that the storage medium can be used to program, configure, and/or adapt a general purpose computer with the instructions/code stored thereon. These instructions may take the form of executable code, which is executable by the computer system 2100 and/or may take the form of source and/or installable code, which, upon compilation and/or installation on the computer system 2100 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, single board computers, FPGAs, ASICs, and SoCs) may also be used, and/or particular elements may be implemented in hardware, software (including portable software, such as applets, etc.), or both. Further, connection to other computing devices such as network input/output devices may be employed.

As mentioned above, in one aspect, some embodiments may employ a computer or hardware system (such as the computer system 2100) to perform methods in accordance with various embodiments of the invention. According to a set of embodiments, some or all of the procedures of such methods are performed by the computer system 2100 in response to processor 2110 executing one or more sequences of one or more instructions (which may be incorporated into the operating system 2140 and/or other code, such as an application program 2145 or firmware) contained in the working memory 2135. Such instructions may be read into the working memory 2135 from another computer readable medium, such as one or more of the storage device(s) 2125. Merely by way of example, execution of the sequences of instructions contained in the working memory 2135 may cause the processor(s) 2110 to perform one or more procedures of the methods described herein.

The terms “machine readable medium” and “computer readable medium,” as used herein, refer to any medium that participates in providing data that causes a machine to operate in a specific fashion. In an embodiment implemented using the computer system 2100, various computer readable media may be involved in providing instructions/code to processor(s) 2110 for execution and/or may be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 2125. Volatile media includes, without limitation, dynamic memory, such as the working memory 2135. In some alternative embodiments, a computer readable medium may take the form of transmission media, which includes, without limitation, coaxial cables, copper wire and fiber optics, including the wires that comprise the bus 2105, as well as the various components of the communication subsystem 2130 (and/or the media by which the communications subsystem 2130 provides communication with other devices). In an alternative set of embodiments, transmission media can also take the form of waves (including, without limitation, radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 2110 for execution. Merely by way of example, the instructions may initially be carried on a magnetic disk and/or optical disc of a remote computer. A remote computer may load the instructions into its dynamic memory and send the instructions as signals over a transmission medium to be received and/or executed by the computer system 2100. These signals, which may be in the form of electromagnetic signals, acoustic signals, optical signals, and/or the like, are all examples of carrier waves on which instructions can be encoded, in accordance with various embodiments of the invention.

The communications subsystem 2130 (and/or components thereof) generally receives the signals, and the bus 2105 then may carry the signals (and/or the data, instructions, etc. carried by the signals) to the working memory 2135, from which the processor(s) 2110 retrieves and executes the instructions. The instructions received by the working memory 2135 may optionally be stored on a storage device 2125 either before or after execution by the processor(s) 2110.

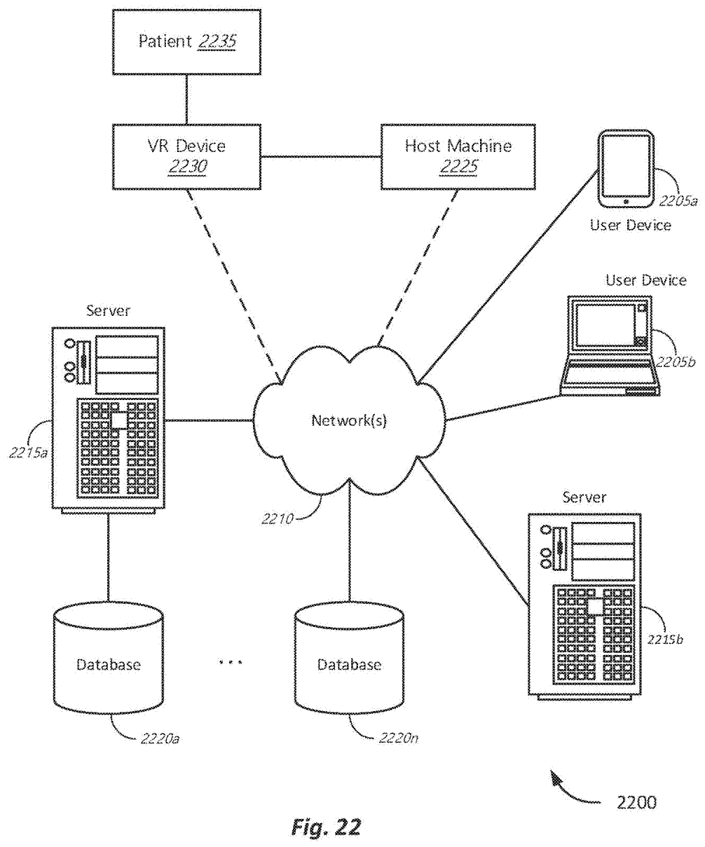

FIG. 22 is a schematic block diagram illustrating system 2200 of networked computer devices, in accordance with various embodiments. The system 2200 may include one or more user devices 2205. A user device 2205 may include, merely by way of example, desktop computers, single-board computers, tablet computers, laptop computers, handheld computers, edge devices, wearable devices, and the like, running an appropriate operating system. User devices 2205 may further include external devices, remote devices, servers, and/or workstation computers running any of a variety of operating systems. A user device 2205 may also have any of a variety of applications, including one or more applications configured to perform methods provided by various embodiments, as well as one or more office applications, database client and/or server applications, and/or web browser applications. Alternatively, a user device 2205 may include any other electronic device, such as a thin-client computer, Internet-enabled mobile telephone, and/or personal digital assistant, capable of communicating via a network (e.g., the network(s) 2210 described below) and/or of displaying and navigating web pages or other types of electronic documents. Although the exemplary system 2200 is shown with two user devices 2205a-2205b, any number of user devices 2205 may be supported.

Certain embodiments operate in a networked environment, which can include a network(s) 2210. The network(s) 2210 can be any type of network familiar to those skilled in the art that can support data communications, such as an access network, core network, or cloud network, and use any of a variety of commercially-available (and/or free or proprietary) protocols, including, without limitation, MQTT, CoAP, AMQP, STOMP, DDS, SCADA, XMPP, custom middleware agents, Modbus, BACnet, NCTIP, Bluetooth, Zigbee/Z-wave, TCP/IP, SNA™ IPX™ and the like. Merely by way of example, the network(s) 2210 can each include a local area network (“LAN”), including, without limitation, a fiber network, an Ethernet network, a Token-Ring™ network and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, and/or any other wireless protocol; and/or any combination of these and/or other networks. In a particular embodiment, the network may include an access network of the service provider (e.g., an Internet service provider (“ISP”)). In another embodiment, the network may include a core network of the service provider, backbone network, cloud network, management network, and/or the Internet.

Embodiments can also include one or more server computers 2215. Each of the server computers 2215 may be configured with an operating system, including, without limitation, any of those discussed above, as well as any commercially (or freely) available server operating systems. Each of the servers 2215 may also be running one or more applications, which can be configured to provide services to one or more clients 2205 and/or other servers 2215.

Merely by way of example, one of the servers 2215 may be a data server, a web server, orchestration server, authentication server (e.g., TACACS, RADIUS, etc.), cloud computing device(s), or the like, as described above. The data server may include (or be in communication with) a web server, which can be used, merely by way of example, to process requests for web pages or other electronic documents from user computers 2205. The web server can also run a variety of server applications, including HTTP servers, FTP servers, CGI servers, database servers, Java servers, and the like. In some embodiments of the invention, the web server may be configured to serve web pages that can be operated within a web browser on one or more of the user computers 2205 to perform methods of the invention.

The server computers 2215, in some embodiments, may include one or more application servers, which can be configured with one or more applications, programs, web-based services, or other network resources accessible by a client. Merely by way of example, the server(s) 2215 can be one or more general purpose computers capable of executing programs or scripts in response to the user computers 2205 and/or other servers 2215, including, without limitation, web applications (which may, in some cases, be configured to perform methods provided by various embodiments). Merely by way of example, a web application can be implemented as one or more scripts or programs written in any suitable programming language, such as Java™, C, C#™ or C++, and/or any scripting language, such as Perl, Python, or TCL, as well as combinations of any programming and/or scripting languages. The application server(s) can also include database servers, including, without limitation, those commercially available from Oracle™, Microsoft™, Sybase™ IBM™, and the like, which can process requests from clients (including, depending on the configuration, dedicated database clients, API clients, web browsers, etc.) running on a user computer, user device, or customer device 2205 and/or another server 2215.

In accordance with further embodiments, one or more servers 2215 can function as a file server and/or can include one or more of the files (e.g., application code, data files, etc.) necessary to implement various disclosed methods, incorporated by an application running on a user computer 2205 and/or another server 2215. Alternatively, as those skilled in the art will appreciate, a file server can include all necessary files, allowing such an application to be invoked remotely by a user computer, user device, or customer device 2205 and/or server 2215.

It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 2220a-2220n (collectively, “databases 2220”). The location of each of the databases 2220 is discretionary: merely by way of example, a database 2220a may reside on a storage medium local to (and/or resident in) a server 2215a (or alternatively, user device 2205). Alternatively, a database 2220n can be remote so long as it can be in communication (e.g., via the network 2210) with one or more of these. In a particular set of embodiments, a database 2220 can reside in a storage-area network (“SAN”) familiar to those skilled in the art. In one set of embodiments, the database 2220 may be a relational database configured to host one or more data lakes collected from various data sources. The databases 2220 may include SQL, no-SQL, and/or hybrid databases, as known to those in the art. The database may be controlled and/or maintained by a database server.

The system 2200 may further include host machine 2225 coupled to a VR device 2230. The VR device 2230 may in turn be coupled to the patient 2235. As previously described with respect to the above embodiments, the host machine 2225 may be configured to provide eating behavior training to the patient 2235 via the VR device 2230.

Experimental Results

Experimental results from a VR system, according to at least one embodiment, are provided. Once the written consent had been signed, patients in the study were seated in front of a table and fitted with VR equipment and the VR application was started. The VR application had been calibrated so that a virtual table identical to the real table was present in the VR environment. Before the patient performed any actions in the VR application, voice and video recording was initiated.

The VR session began with a tutorial that helped the patient familiarize with the equipment and the virtual environment. The virtual items included serving utensils, food (potatoes, meatballs, lingonberry jam, gravy and drinking water), and a smart device. The smart device had instructions that guided the user at each step on the tasks to perform in order to meet their goals. During the first step of the tutorial, patients were informed that upon completion of a tutorial step they should press the ‘Next’ button on the smart device, using the hand controls, to move to the next step. During the second step they used the hand controls as hands in the VR environment to pick up the plate from the table and place it on a scale. On completion of this step, the patient pressed the ‘Next’ button that was on the top corner of the smart device.

A pan with meatballs and a wooden spoon then appeared. The patient served as many meatballs as they liked using the wooden spoon. On completion of this task, the patient pressed the ‘Next’ button and a bowl with potatoes and a fork appeared on the table. The patient picked up the fork and served as many potatoes as they wanted onto their plate. Once this step was completed, the patient moved to the next step by clicking on the ‘Next’ button. A gravy boat with gravy in it appeared. The patient's task was then to select this boat, pick it up and serve themselves gravy and place the gravy boat back on the table. To proceed to the subsequent step, the patient selected the ‘Next’ button. A smaller bowl with lingonberry jam and a small spoon appeared on the table. The patient picked up the spoon and served as much lingonberry jam as they wanted.

After completion of this step and selection of the ‘Next’ button. A jug of water and a glass appeared, and the patients were asked to serve themselves as much water as they desired. After selecting the ‘Next button,’ a knife and a fork appeared beside the plate. The patient's task was to pick up the knife and the fork, and use them to manipulate and eat the food. The patient ended the tutorial session when they felt confident with the items within the VR environment.

The patient then began a VR training meal, during which they first served the virtual food as they did in the tutorial. Within this module, patients were required to serve a specific amount; the amount of food was indicated as a percentage, and the patients filled their plates until the smart device indicated “100%.” The patients then initiated the meal by pressing on the ‘start’ button on the smart device. During the meal, patients received feedback on the eating rate they should maintain by aid of a training curve visible on the smart device. If they ate too fast or slow a message appeared informing them they should slow down or speed up their eating, respectively. During the meal patients also rated satiety once every minute. Due to the application being a demo version the patients had only three minutes to eat the food, while in a clinical setting meals are at least 10 minutes long.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to certain structural and/or functional components for ease of description, methods provided by various embodiments are not limited to any single structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in sequentially for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a specific structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to one embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A system comprising:
a virtual reality device configured to display a virtual reality interface to a patient;
a computing system coupled to the virtual reality device, the computing system comprising:
a) a processor; and
b) a computer readable medium in communication with the processor, the computer readable medium having encoded thereon a set of instructions executable by the processor to:
1) accept an input of one or more patient eating behaviors;
2) evaluate the input of one or more patient eating behaviors to determine a patient eating disorder;
3) determine a target eating behavior based, at least in part, on the patient eating disorder;
4) generate a virtual scenario for training the patient to accomplish the target eating behavior;
5) present the virtual scenario to the patient through the virtual reality interface, wherein the virtual scenario allows the patient to virtually accomplish the target eating behavior;
6) monitor one or more patient actions within the virtual scenario through the virtual reality interface; and
7) provide real-time feedback to the patient, through the virtual reality interface, based on the one or more patient actions within the virtual scenario, said real-time feedback being configured to encourage the target eating behavior.

2. The system of claim 1, wherein the set of instructions is further executable by the processor to utilize at least one of artificial intelligence functionality or machine learning functionality to perform at least one of:
a) evaluating the input of one or more patient eating behaviors;
b) determining the patient eating disorder; and
c) determining the target eating behavior.

3. The system of claim 1, wherein the virtual reality interface comprises a visual output, an audible output and at least one of a tactile and an olfactory output.

4. The system of claim 1, wherein the virtual reality interface comprises at least one biofeedback detector.

5. The system of claim 1 wherein the at least one biofeedback detector comprises at least one of an eye motion detector, a pupil dilation detector, a blink rate detector, a skin temperature detector, a perspiration level detector, a breathing rate detector, a cardiac pulse rate detector, a blood oxygen level detector, and a blood pressure detector.

6. The system of claim 1, wherein the virtual scenario presents one or more successive eating approximations configured to initiate the target eating behavior in the patient.

7. The system of claim 6, wherein the one or more successive eating approximations includes at least one of setting a table, pouring a drink, placing a food on a plate, picking up the food with a fork, and smelling the food.

8. The system of claim 1, wherein the set of instructions is further executable by the processor to generate, from the input of one or more patient eating behaviors, a list of fear or forbidden foods from the patient, and wherein the virtual scenario is generated based, at least in part, on the list of fear or forbidden foods from the patient.

9. The system of claim 8, wherein the virtual scenario includes a food selection scenario, and wherein the food selection scenario is configured to allow the patient to virtually select at least one item from the list of fear or forbidden foods for consumption.

10. The system of claim 1, wherein the virtual scenario is configured to present one or more virtual human guides to the patient within the virtual scenario, wherein the one or more virtual human guides model the target eating behavior within virtual scenario.

11. The system of claim 1, wherein the set of instructions is further executable by the processor to determine, based on the one or more patient actions, whether the patient eating disorder is in remission.

12. The system of claim 1, wherein the set of instructions is further executable by the processor to present a stimulus in the virtual scenario to elicit a reward response in the patient.

13. A method comprising:
a) using a computing system to evaluate an input of one or more patient eating behaviors;
b) determining, with the computing system, a patient eating disorder based at least in part upon the input;
c) determining, with the computing system, a target eating behavior based, at least in part, upon the patient eating disorder;
d) generating, with the computing system, a virtual scenario for training the patient to accomplish the target eating behavior;
e) presenting the virtual scenario to the patient, through a virtual reality interface, wherein the virtual scenario allows the patient to interact virtually with the computing system to virtually accomplish the target eating behavior;
f) monitoring, through the virtual reality interface, one or more patient actions within the virtual scenario; and
g) providing real-time feedback to the patient, through the virtual reality interface, based upon the one or more patient actions within the virtual scenario, said feedback being configured to encourage the target eating behavior.

14. The method of claim 13 further comprising utilizing at least one of artificial intelligence functionality or machine learning functionality to perform at least one of:
a) evaluating the input of one or more patient eating behaviors;
b) determining the patient eating disorder; and
c) determining the target eating behavior.

15. The method of claim 13 further comprising generating and presenting the virtual scenario through the virtual reality interface to include a visual output, an audible output and at least one of a tactile output and an olfactory output.

16. The method of claim 13 further comprising detecting biofeedback from the patient while monitoring the one or more patient actions within the virtual scenario.

17. The method of claim 16, wherein the biofeedback comprises at least one of eye motion, pupil dilation, blink rate, skin temperature, perspiration level, breathing rate, cardiac pulse rate, blood oxygen level and blood pressure.

18. The method of claim 13, further comprising presenting one or more successive eating approximations configured to initiate the target eating behavior in the patient.

19. The method of claim 18 wherein the one or more successive eating approximations includes at least one of setting a table, pouring a drink, placing a food on a plate, picking up the food with a fork, and smelling the food.

20. The method of claim 13 further comprising:
a) evaluating the input to generate a list of fear or forbidden foods for the patient; and
b) generating the virtual scenario, at least in part, upon the list of fear or forbidden foods; wherein the virtual scenario includes a food selection scenario, and wherein the food selection scenario is configured to allow the patient to virtually select at least one item from the list of fear or forbidden foods for consumption.

21. The method of claim 13, further comprising:
generating the virtual scenario to include a virtual human guide configured to model the target eating behavior within the virtual scenario.

22. The method of claim 13, further comprising presenting a stimulus in the virtual scenario to elicit a reward response in the patient.

23. The method of claim 13, further comprising determining at least in part upon the one or more patient actions whether the patient eating disorder is in remission.

24. The method of claim 13 further comprising presenting a stimulus in the virtual scenario to elicit a reward response in the patient.

25. An apparatus, comprising:
a) at least one processor; and
b) a non-transitory computer readable medium communicatively coupled to the at least one processor, the non-transitory computer readable medium having stored thereon computer software comprising a set of instructions that, when executed by the at least one processor, causes the apparatus to:
1) accept an input of one or more patient eating behaviors;
2) evaluate the input of one or more patient eating behaviors to determine a patient eating disorder;
3) determine a target eating behavior based, at least in part, on the determined patient eating disorder;
4) generate a virtual scenario for training a patient to accomplish the target eating behavior;
5) present the virtual scenario to the patient, wherein the virtual scenario allows the patient to virtually accomplish the target eating behavior;
6) monitor one or more patient actions within the virtual scenario; and
7) provide real-time feedback to the patient based on the one or more patient actions within the virtual scenario, said feedback being configured to encourage the target eating behavior.

26. The apparatus of claim 25, wherein the set of instructions is further executable by the processor to utilize at least one of artificial intelligence functionality or machine learning functionality to perform at least one of:
a) evaluating the input of the one or more patient eating behaviors;
b) determining the patient eating disorder; and
c) determining the target eating behavior.

27. The apparatus of claim 25, wherein the set of instructions is further executable by the processor to provide a visual output, an audible output and at least one of a tactile and an olfactory output.

28. The apparatus of claim 25, wherein the set of instructions is further executable by the processor to accept biofeedback data from at least one biofeedback detector.

29. The apparatus of claim 28, wherein the set of instructions is further executable by the processor to accept the biofeedback data from at least one of an eye motion detector, a pupil dilation detector, a blink rate detector, a skin temperature detector, a perspiration level detector, a breathing rate detector, a cardiac pulse rate detector, a blood oxygen level detector, and a blood pressure detector.

30. The apparatus of claim 25, wherein the virtual scenario includes one or more successive eating approximations configured to initiate the target eating behavior in the patient.

31. The apparatus of claim 30, wherein the one or more successive eating approximations includes at least one of setting a table, pouring a drink, placing a food on a plate, picking up the food with a fork, and smelling the food.

32. The apparatus of claim 25, wherein the set of instructions is further executable by the processor to generate, from the input of one or more patient eating behaviors, a list of fear or forbidden foods from the patient, and wherein the virtual scenario is generated based, at least in part, on the list of fear or forbidden foods from the patient.

33. The apparatus of claim 32, wherein the virtual scenario includes a food selection scenario, and wherein the food selection scenario is configured to allow the patient to virtually select at least one item from the list of fear or forbidden foods for consumption.

34. The apparatus of claim 25, wherein the virtual scenario is configured to present one or more virtual human guides to the patient within the virtual scenario, wherein the virtual human guides model the target eating behavior within virtual scenario.

35. The apparatus of claim 25, wherein the set of instructions is further executable by the processor to determine, based on the one or more patient actions, whether the patient eating disorder is in remission.

36. The apparatus of claim 25, wherein the set of instructions is further executable by the processor to present a stimulus in the virtual scenario to elicit a reward response in the patient.
